SEQUENCE LISTING (6 sequences)

SEQ ID NO: 1 - DNA, 39 bp, synthetic construct
  source 1..39: mol_type = other DNA
cgtggagctacagtggtaaagcaggtgaagaaatgcagt

SEQ ID NO: 2 - DNA, 21 bp, synthetic construct
  source 1..21: mol_type = other DNA
gtaactgcatttcttcacctg

SEQ ID NO: 3 - DNA, 18 bp, synthetic construct
  source 1..18: mol_type = other DNA
cactgtagctccacgacc

SEQ ID NO: 4 - DNA, 39 bp, synthetic construct
  source 1..39: mol_type = other DNA
cgtggagttacagtggtaaagcaggtgaagaaatgcagt

SEQ ID NO: 5 - DNA, 39 bp, synthetic construct
  source 1..39: mol_type = other DNA
cgtggagttacagtggtaaagcaggtgtagaaatgcagt

SEQ ID NO: 6 - DNA, 39 bp, synthetic construct
  source 1..39: mol_type = other DNA
cgtggagttacagcggtaaagcaggtgtagaaatgcagt

(19) United States
(12) Patent Application Publication
(10) Pub. No.: US 2024/0011911 A1
(43) Pub. Date: Jan. 11, 2024

(54) LECTROCHEMILUMINESCENCE IMMUNOASSAY-NUCLEIC ACID TESTING SYNCHRONOUS MULTICOMPONENT ANALYSIS METHOD BASED ON SPECTRAL RESOLUTION PRINCIPLE

(71) Applicant: SHANDONG UNIVERSITY, Jinan (CN)

(72) Inventors: Guizheng ZOU, Jinan (CN); Xuwen GAO, Jinan (CN)

(21) Appl. No.: 18/162,234

(22) Filed: Jan. 31, 2023

(30) Foreign Application Priority Data
Jul. 5, 2022 (CN) 2022107819757

(51) Int. Cl. G01N 21/76; G01N 33/543; G01N 27/30

(52) U.S. Cl. CPC G01N 21/76; G01N 33/54306; G01N 27/30

(57) ABSTRACT

The present invention relates to a multiplexing analysis strategy based on spectrum-resolved electrochemiluminescence (ECL) for simultaneous immunoassay and nucleic acid detection. The present invention employs Au nanoclusters (Au NCs) with ECL maximum emissiom wavelength of 485 nm and water-soluble CulnS₂@ZnS nanocrystals (CIS@ZnS NCs) with ECL maximum emissiom wavelength of 775 nm as biomarkers to fabricate the multiplexing ECL sensor for simultaneous detection of protein CEA and nucleic acid p53, which breakthrough the reported ECL multiplexing sensor that is only capable of detecting multiple proteins or multiple nucleic acids, and avoids the time-consuming DNA amplification process.

### REFERENCE TO AN ELECTRONIC SEQUENCE LISTING

The contents of the electronic sequence listing (230161.xml; Size: 5,519 bytes; and Date of Creation: Apr. 21, 2023) is herein incorporated by reference in its entirety.

### CROSS REFERENCES

This application claims priority to Chinese Patent Application Ser. No. CN2022107819757 filed on 5 Jul. 2022.

### FIELD

The present invention relates to an electrochemiluminescence immunoassay-nucleic acid testing synchronous multicomponent analysis method based on spectral resolution principle, and belongs to the technical field of electrochemiluminescence assay.

### BACKGROUND

In the context that highly infectious severe acute respiratory syndrome coronavirus 2 (SARS-CoV-2) has become a global epidemic trend, quick detection of viral strains is of great value for timely isolation of infected individuals and interruption of virus transmission. Nucleic acid testing is the gold standard for diagnosis of SARS-CoV-2. Nucleic acid testing is usually implemented based on reverse transcription-polymerase chain reaction (RT-PCR) amplification and reverse transcription-loop-mediated isothermal amplification (RT-LAMP), and has the disadvantages of complicated equipment requirements, time-consuming testing, accuracy of testing results being affected by virus mutation and the like. Academic and industrial circles have began to introduce immunoassay into the field of SARS-CoV-2 testing, taken the antigen and antibody testing as auxiliary and supplementary means of diagnosis of SARS-CoV-2 infection, and applied it to the tracing of outbreak in clusters. No nucleic acid testing-immunoassay synchronous multicomponent analysis technology is available yet in the academic and industrial circles although a combination of nucleic acid testing and immunoassay is of great value for interruption of virus diffusion.

Electrochemiluminescence (ECL) in-vitro diagnosis technology has the remarkable features of high sensitivity and simple device to provide potential feasibility for avoiding a cumbersome amplification procedure of nucleic acid testing. The development of ECL multicomponent analysis technology has made it possible to test two antigens or nucleic acid fragments simultaneously. The band identification type two-component ECL sensor established by He realizes simultaneous testing of the two nucleic acids wild type p53 and mutant p53 (Anal. Chem. 2018, 90, 5474-5480); the band identification type two-component ECL immunosensor established by Zhang realizes simultaneous testing of the two proteins AFP and CA125 (Biosens. Bioelectron. 2018, 115, 77-82). Chinese Patent Document No. CN106124487A provides an ECL three-component immunosensor based on spectral resolution principle, which realizes simultaneous testing of three antigens by taking CdSe quantum dots and CdTe quantum dots as labels.

So far, both band identification type and spectral resolution type ECL multicomponent analysis methods are mainly based on II-VI group nanomaterials, and the synchronous testing of specific antigens and nucleic acid fragments cannot be implemented synchronously. The ECL analysis method that enables synchronous implementation of immunoassay and nucleic acid testing is still in a blank state.

### SUMMARY

The present invention provides an ECL immunoassay-nucleic acid testing synchronous multicomponent analysis method based on spectral resolution principle in view of the disadvantages in the prior art, in particular to the status that it is still impossible to implement synchronous testing of specific antigens and nucleic acid fragments based on the spectral resolution principle.

In the present invention, a two-color and two-component spectral resolution type ECL sensor is established using water-soluble Au nanoclusters (Au NCs) with the maximum radiation wavelength of ECL at 485 nm and water-soluble CIS@ZnS NCs quantum dots with the maximum radiation wavelength of ECL at 775 nm as labels, and the synchronous testing of protein CEA and nucleic acid p53 is realized, which breaks through the limitation of the reported multicomponent ECL sensor that it is difficult to implement nucleic acid testing and immunoassay synchronously; moreover, the time-consuming DNA amplification process is avoided effectively.

Terminology:

Primary antibody (CEA-Ab₁): A primary antibody (Ab₁) used herein refers to a corresponding antibody to a carcinoembryonic antigen (CEA), and a monoclonal antibody corresponding to the antigen can obtain a better effect in the present invention.

Secondary antibody (CEA-Ab₂): A secondary antibody used herein refers to a corresponding secondary antibody to the CEA antigen and the primary antibody.

Target DNA (Tₚ₅₃): A target DNA used herein refers to a specific gene (single-stranded).

Captured DNA (Cₚ₅₃): A captured DNA used herein refers to a complementary strand of a segment of the above-mentioned specific gene, which is labeled with a sulfhydryl group.

Probe DNA (Pₚ₅₃): A probe DNA used herein refers to a complementary strand of a segment of the above-mentioned specific gene, which is labeled with an amino group, with a nucleotide sequence different from that of the captured DNA.

The present invention is implemented through the following technical solution:

An ECL immunoassay-nucleic acid testing synchronous multicomponent analysis method based on spectral resolution principle, including the following steps:
1) establishing an ECL multicomponent analysis sensor for synchronous implementation of immunoassay and nucleic acid testing, wherein the maximum radiation wavelengths of the ECL multicomponent analysis sensor are at 485 nm and 775 nm respectively; the ECL multicomponent analysis sensor includes a secondary antibody labeled with Au NCs (Ab₂|Au NCs) and a probe DNA fragment labeled with CIS@ZnS NCs quantum dots (Pₚ₅₃|CIS@ZnS NCs), CEA-Ag and Tₚ₅₃, Au|MPA-Ab₁ and Au|MPA-Cₚ₅₃, and the ECL multicomponent analysis sensor (Au|MPA−Cₚ₅₃^{Ab1}<Tₚ₅₃^{Ag}>Pₚ₅₃^{Ab2}|CIS_{@ZnSNCs}^{Au NCs}) is established based on selectivity of immune and nucleic acid reactions;
2) producing ECL by taking the ECL multicomponent analysis sensor as a working electrode, a platinum electrode as a counter electrode, and an Ag/AgCl electrode as a reference electrode when cyclic voltammetry is used for driving in a Hepes buffer solution containing 5-20 mM hydrazine hydrate;
3) collecting all photons in the whole process of ECL by means of exposure imaging, and obtaining a total spectrum by means of radiation of all the photons based on dispersive ECL; drawing a working curve of CEA testing according to a relationship between the maximum radiation intensity at the maximum radiation wavelength of 485 nm on a spectral curve and the concentration of a standard antigen solution; drawing a working curve of Tₚ₅₃ testing according to a relationship between the maximum radiation intensity at the maximum radiation wavelength of 775 nm on the spectral curve and the concentration of Tₚ₅₃;
4) using a sample to be tested to establish an ECL sensor for synchronous implementation of immunoassay and nucleic acid testing according to step 1), performing an ECL spectral test according to the methods in step 2) and step 3), and synchronously testing the concentrations of the antigen and target DNA in the sample solution to be tested according to a light intensity signal and a working curve at the maximum radiation wavelength on the obtained ECL spectral curve.

According to the present invention, preferably, in step 2) and step 4), when cyclic voltammetry scanning is performed, a scanning voltage ranges from 0 V to 1.6 V, a number of scanning turns is 1 to 3, and a scanning speed is 40 to 60 mV/s. Corresponding ECL radiations are generated respectively when the Au NCs and CIS@ZnS NCs fixed to the surface of the working electrode are driven by cyclic voltammetry.

According to the present invention, preferably, in step 1), the method for establishing the ECL multicomponent analysis sensor for synchronous implementation of immunoassay and nucleic acid testing is as follows:
a. using a cleaned and activated Au electrode as a working electrode, and labeling both CEA-Ab₁ and Cₚ₅₃ on the surface of the electrode to obtain a double-labeled Au electrode;
b. labeling the water-soluble Au NCs with CEA-Ab₂ to obtain Ab₂|Au NCs; labeling the water-soluble CIS@ZnS NCs with a probe DNA to obtain Pₚ₅₃|CIS@ZnS NCs;
c. adding CEA-Ag and Tₚ₅₃ dropwise to the surface of the double-labeled Au electrode, incubating the mixture at room temperature, adding the Ab₂|Au NCs and Pₚ₅₃|CIS@ZnS NCs obtained in step b dropwise to the surface of the electrode and incubating the mixture; grafting Ab₂|Au NCs and Pₚ₅₃|CIS@ZnS NCs to the surface of the working electrode in a form of immune complex formation to obtain an ECL multicomponent analysis sensor for synchronous implementation of immunoassay and nucleic acid testing.

According to the present invention, preferably, in step a, the preparation steps of the double-labeled Au electrode are as follows:
(1) soaking a cleaned Au electrode in 5-20 mM mercaptopropionic acid overnight, and bonding MPA to the surface of the electrode through an Au—S bond;
(2) adding 10 μL of 10 mg/mL 1-ethyl-(3-dimethylaminopropionic acid) carbodiimide hydrochloride (EDC) and 10 mg/mL hydroxysuccinimide (NHS) dropwise to the surface of the modified electrode obtained in step (1), activating the mixture for 30 min, cleaning the electrode, and removing unreacted EDC and NHS;
(3) mixing an aqueous solution of CEA-Ab₁ and an aqueous solution of Cₚ₅₃, adding the mixture to the surface of the activated electrode, incubating the mixture for 2-4 h, adding the mixture to unreacted active sites on a BSA closed electrode, and cleaning the electrode to obtain a double-labeled Au electrode.

According to the present invention, preferably, the concentration of the aqueous solution of CEA-Ab₁ is 8-15 μg/mL with an added amount of 8-15 μL, and the concentration of the aqueous solution of Cₚ₅₃ is 8-15 μM with an added amount of 8-15 μL.

According to the present invention, preferably, in step b for establishing the ECL multicomponent analysis sensor for synchronous implementation of immunoassay and nucleic acid testing, the synthesis steps of the Ab₂|Au NCs are as follows:
1) activating carboxylic acid groups on the surface of water-soluble Au NCs;
2) enabling the secondary antibody to react with the carboxylic acid groups on the surface of the water-soluble Au NCs treated in step 1) to obtain a secondary antibody corresponding to the antigen labeled by the water-soluble Au NCs.

According to the present invention, preferably, the specific preparation steps of the Ab₂|Au NCs are as follows:

dissolving purified Au NCs in 1 mL of 0.1 M pH 6.0 phosphate buffered solution (PBS) containing 10 mg/mL EDC and 10 mg/mL NHS, activating the mixture for 30 min, performing centrifugal purification, and dispersing the mixture in 1 mL of pH 7.4 0.1 M PBS to obtain activated Au NCs; adding 8-15 μL of 8-15 μg/mL aqueous solution of CEA-Ab₂, incubating the mixture at a constant temperature of 37C for 3-5 h, adding 20 L of bovine serum albumin (BSA), sealing for 30 min, centrifuging the mixture, and collecting sediments to obtain Ab₂|Au NCs.

According to the present invention, preferably, the above-mentioned water-soluble Au NCs are prepared from chloroauric acid as an Au source, mercaptopropionic acid as a stabilizer and zinc acetate as an aggregation inducer.

According to the present invention, preferably, the synthesis steps of the water-soluble Au NCs are as follows:
(1) taking 35.5 μL of 100 mg/mL HAuCl₄·3H₂O, and adding 2.5 mL of deionized water;
(2) adding 50 μL of mercaptopropionic acid to the mixture in step (1), and stirring the mixture for 15 min;
(3) adding 430 μL of 1 M sodium hydroxide to the mixture in step (2), and adjusting the pH to 8.5;
(4) adding 0.5 mL of 0.1 M zinc acetate to the mixture in step (3), stirring the mixture at room temperature for a reaction for 6 h, washing and purifying the reaction product with isopropanol after the reaction is completed to obtain water-soluble Au NCs.

According to the present invention, preferably, in step b for establishing the ECL multicomponent analysis sensor for synchronous implementation of immunoassay and nucleic acid testing, the synthesis steps of the Pₚ₅₃|CIS@ZnS NCs are as follows:
1) activating carboxylic acid groups on the surface of water-soluble CIS@ZnS NCs;
2) making Pₚ₅₃ react with the carboxylic acid groups on the surface of the activated water-soluble CIS@ZnS NCs to obtain a probe DNA corresponding to a target DNA labeled with the water-soluble CIS@ZnS NCs.

According to the present invention, preferably, the specific preparation steps of the Pₚ₅₃|CIS@ZnS NCs are as follows:

dissolving purified CIS@ZnS NCs in 1 mL of 0.1 M pH 6.0 PBS containing 10 mg/mL EDC and 10 mg/mL NHS, activating the mixture for 30 min, performing centrifugal purification, and dispersing the mixture in 1 mL of pH 7.4 0.1 M PBS to obtain activated CIS@ZnS NCs; adding 8-15 μL of 8-15 μM aqueous solution of Pₚ₅₃, incubating the mixture at a constant temperature of 37° C. for 3-5 h, connecting an amino group at one end of the probe DNA and a carboxyl group on the surface of CIS@ZnS NCs through an amidation reaction, adding 20 μL of BSA, sealing for 30 min, centrifuging the mixture, and collecting sediments to obtain Pₚ₅₃|CIS@ZnS NCs.

According to the present invention, preferably, the above-mentioned water-soluble CIS@ZnS NCs are water-soluble CuInS₂@ZnS NCs prepared from CuCl₂·2H₂O as a Cu source, InCl₃·4H₂O as an In source, and sodium citrate and captopril as stabilizers.

According to the present invention, preferably, the synthesis steps of the water-soluble CIS@ZnS NCs are as follows:
(1) dissolving 0.0022 g of captopril, 0.01 g of NaOH, 0.0471 g of sodium citrate, 0.0017 g of CuCl₂·2H₂O, 0.0117 g of InCl₃·4H₂O and 0.0048 g of Na₂S in 20 mL of deionized water successively under a stirring condition, heating the mixture to 95° C., and holding for 45 min;
(2) adding 0.177 g of Zn(CH₃COO)₂ and 0.061 g of thiourea to the mixture in step (1);
(3) adding isopropanol to the mixture in step (2) for washing and purification to obtain water-soluble CIS@ZnS NCs.

A specific method for establishing a preferred embodiment of the present invention: ECL multicomponent analysis sensor for synchronous implementation of immunoassay and nucleic acid testing, is as follows:
a. soaking a cleaned Au electrode in 10 mM mercaptopropionic acid overnight, and bonding MPA to the surface of the electrode through an Au—S bond;
b. adding 10 μL of 10 mg/mL EDC and 10 mg/mL NHS dropwise to the surface of the modified electrode obtained in a, activating the mixture for 30 min, cleaning the electrode, and removing unreacted EDC and NHS;
c. mixing 10 μL of 10 μg/mL aqueous solution of CEA-Ab₁ and 10 μL of 10 μM aqueous solution of Cₚ₅₃, adding the mixture to the surface of the activated electrode obtained in step b, incubating the mixture for 3 h, adding the mixture to unreacted active sites on a BSA closed electrode, and cleaning the electrode to obtain a double-labeled Au electrode;
d. dissolving purified Au NCs in 1 mL of 0.1 M pH 6.0 PBS containing 10 mg/mL EDC and 10 mg/mL NHS, activating the mixture for 30 min, performing centrifugal purification, and dispersing the mixture in 1 mL of pH 7.4 0.1 M PBS to obtain activated Au NCs; adding 10 μL of 10 μg/mL aqueous solution of CEA-Ab₂, incubating the mixture at a constant temperature of 37C for 3-5 h, adding 20 μL of BSA, sealing for 30 min, centrifuging the mixture, and collecting sediments to obtain Ab₂|Au NCs;
e. dissolving purified CIS@ZnS NCs in 1 mL of 0.1 M pH 6.0 PBS containing 10 mg/mL EDC and 10 mg/mL NHS, activating the mixture for 30 min, performing centrifugal purification, and dispersing the mixture in 1 mL of pH 7.4 0.1 M PBS to obtain activated CIS@ZnS NCs; adding 10 μL of 10 μM aqueous solution of probe DNA, incubating the mixture at a constant temperature of 37° C. for 3-5 h, adding 20 L of BSA, sealing for 30 min, centrifuging the mixture, and collecting sediments to obtain Pₚ₅₃|CIS@ZnS NCs;
f. adding CEA-Ag and Tₚ₅₃ dropwise to the surface of the double-labeled Au electrode, incubating the mixture at room temperature for 90 min, cleaning the electrode, mixing and adding the Ab₂|Au NCs and Pₚ₅₃|CIS@ZnS NCs dropwise to the surface of the electrode and incubating the mixture for 1 h; grafting and fixing Ab₂|Au NCs and Pₚ₅₃|CIS@ZnS NCs in a form of immune complex formation to the surface of the working electrode to obtain an ECL sensor for synchronous implementation of immunoassay and nucleic acid testing.

According to the present invention, preferably, when the above-mentioned added BSA is sealed, the volume fraction of BSA is 1%.

According to the present invention, preferably, flushing liquid used for cleaning the electrode is 10 mM pH=7.4 PBS.

According to the present invention, preferably, the above-mentioned CEA-Ag and Tₚ₅₃ are added dropwise to the surface of the double-labeled Au electrode in the form of aqueous solutions, the concentration of CEA is 0.3 pg/mL˜50 ng/mL, and the concentration of Tₚ₅₃ is 1 μM˜50 nM.

The immunoassay-nucleic acid testing synchronous ECL multicomponent analysis method based on spectral resolution principle of the present invention is used for testing of both a human carcinoembryonic antigen and wild type P53.

According to the present invention, preferably, the Ab₂|Au NCs and Pₚ₅₃|CIS@ZnS NCs are added to the surface of the electrode dropwise in the form of aqueous solutions for incubation, and the concentration of Ab₂|Au NCs is 10-20 mg/mL; the concentration of Pₚ₅₃|CIS@ZnS NCs is 10-20 μM, and the amounts of Ab₂|Au NCs and Pₚ₅₃|CIS@ZnS NCs shall be sufficient; antigen-antibody interaction and complementary base pairing are formed.

According to the present invention, preferably, in the immunoassay-nucleic acid testing synchronous ECL multicomponent analysis method, specifically:
I: aqueous solutions of CEA-Ag with different standard concentrations and aqueous solutions of Tₚ₅₃ with different standard concentrations are prepared, the ECL sensor for synchronous implementation of immunoassay and nucleic acid testing is established according to the method for establishing an ECL sensor for synchronous implementation of immunoassay and nucleic acid testing using the aqueous solutions of CEA-Ag with different standard concentrations and the aqueous solutions of Tₚ₅₃ with different standard concentrations, and ECL is produced by taking the obtained sensor electrode as a working electrode, a platinum electrode as a counter electrode, and an Ag/AgCl electrode as a reference electrode when cyclic voltammetry is used for driving in a Hepes buffer solution containing 5-20 mM hydrazine hydrate;
II: collecting all photons in the whole process of ECL by means of exposure imaging, and obtaining a total spectrum by means of radiation of all the photons based on dispersive ECL; drawing a working curve of CEA testing according to a relationship between the maximum radiation intensity at the maximum radiation wavelength of 485 nm on a spectral curve and the concentration of standard antigens; drawing a working curve of Tₚ₅₃ testing according to a relationship between the maximum radiation intensity at the maximum radiation wavelength of 775 nm on the spectral curve and the concentration of Tₚ₅₃;
III: an ECL sensor for synchronous implementation of immunoassay and nucleic acid testing is established according to the method for establishing an ECL sensor for synchronous implementation of immunoassay and nucleic acid testing using a target DNA to be tested and CEA-Ag to be tested; ECL is produced by taking the obtained sensor electrode as a working electrode, a platinum electrode as a counter electrode, and an Ag/AgCl electrode as a reference electrode when cyclic voltammetry is used for driving in a Hepes buffer solution containing 5-20 mM hydrazine hydrate; the concentrations of the antigen and target DNA in the sample solution to be tested are synchronously tested according to a light intensity signal and a working curve at the maximum radiation wavelength on the obtained ECL spectral curve.

The present invention uses AuNCs coated with mercaptopropionic acid and CIS@ZnS NCs coated with sodium citrate and captopril as ECL labels respectively; the carboxyl groups on the surface of the AuNCs and CIS@ZnS NCs can be activated by EDC and NHS and grafted to the amino groups on the surface of the secondary antibody and the probe DNA to realize the labeling of the secondary antibody and the probe DNA.

According to the present invention, mercaptopropionic acid is grafted to the surface of the Au electrode as the working electrode by covalent bonding, and the primary antibody is grafted by further activating the carboxyl group of mercaptopropionic acid on the surface of the Au electrode with EDC and NHS.

The beneficial effects of the present invention are as follows:
1. The multicomponent analysis method of the present invention realizes both immunoassay and nucleic acid testing based on ECL for the first time, breaks through the reported limitation of the multicomponent ECL sensor which can only implement the testing of multiple proteins or multiple nucleic acids, and avoids the time-consuming DNA amplification process.
2. The ECL immunoassay-nucleic acid testing synchronous multicomponent analysis method of the present invention realizes the establishment of a two-color and two-component spectral resolution type ECL sensor with fully separated radiation band, and enriches the types of labels of the two-color and two-component ECL sensor by taking Au NCs with the maximum radiation wavelength of ECL at 485 nm and CIS@ZnS NCs quantum dots with the maximum radiation wavelength of ECL at 775 nm as labels and taking Hepes containing hydrazine hydrate as buffer solution.
3. The ECL multicomponent analysis sensor of the present invention is established based on a specific interaction between antigen and antibody and a complementary base pairing principle, and is simple in preparation and operation; immunoassay and nucleic acid testing are carried out in a principle of collection of all photons of ECL radiation and chromatic dispersion into spectra, the ECL signal strengths at the maximum radiation wavelengths of 485 nm and 775 nm cover more than 3 orders of magnitude, the concentration of the tested antigen and target DNA covers 4 orders of magnitude, the CEA and target DNA wild type P53 can be tested sensitively, the linearity of CEA testing ranges from 1 pg/mL to 50 ng/mL with a limit of detection of 0.3 pg/mL, and the linearity of P₅₃ testing ranges from 1 μM to 50 nM with a limit of detection of 0.5 μM.

### BRIEF DESCRIPTION OF THE DRAWINGS

FIG. 1 is an ultraviolet absorption and fluorescence emission spectrogram of the Au NCs prepared in Example 1; the abscissa is wavelength, and the ordinate is absorbance/fluorescence intensity.

FIG. 2 is a fluorescence lifetime diagram of the Au NCs prepared in Example 1; the abscissa is time, and the ordinate is fluorescence intensity.

FIG. 3 is a TEM image of the Au NCs prepared in Example 1.

FIG. 4 is an element distribution diagram of the Au NCs prepared in Example 1; the abscissa is energy, and the ordinate is the number of photons.

FIG. 5 is an infrared spectrogram of the Au NCs prepared in Example 1; the abscissa is wave number, and the ordinate is transmissivity.

FIG. 6 is an ultraviolet absorption and fluorescence emission spectrogram of the CIS@ZnS NCs prepared in Example 1; the abscissa is wavelength, and the ordinate is absorbance/fluorescence intensity.

FIG. 7 is a fluorescence lifetime diagram of the CIS@ZnS NCs prepared in Example 1; the abscissa is time, and the ordinate is fluorescence intensity.

FIG. 8 is a TEM image of the CIS@ZnS NCs prepared in Example 1.

FIG. 9 is an element distribution diagram of the CIS@ZnS NCs prepared in Example 1; the abscissa is energy, and the ordinate is the number of photons.

FIG. 10 is an infrared spectrogram of the CIS@ZnS NCs prepared in Example 1; the abscissa is wave number, and the ordinate is transmissivity.

FIG. 11 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Example 1 is 0.3 pg/mL and the concentration of wild type P₅₃ prepared in Example 1 is 0.5 μM; the abscissa is wavelength, and the ordinate is ECL intensity.

FIG. 12 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Example 2 is 1 pg/mL and the concentration of wild type P₅₃ prepared in Example 2 is 1 μM; the abscissa is wavelength, and the ordinate is ECL intensity.

FIG. 13 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Example 3 is 5 pg/mL and the concentration of wild type P₅₃ prepared in Example 3 is 5 μM; the abscissa is wavelength, and the ordinate is ECL intensity.

FIG. 14 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Example 4 is 50 pg/mL and the concentration of wild type P₅₃ prepared in Example 4 is 50 μM; the abscissa is wavelength, and the ordinate is ECL intensity.

FIG. 15 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Example 5 is 500 pg/mL and the concentration of wild type P₅₃ prepared in Example 5 is 500 μM; the abscissa is wavelength, and the ordinate is ECL intensity.

FIG. 16 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Example 6 is 3,000 pg/mL and the concentration of wild type P₅₃ prepared in Example 6 is 3,000 μM; the abscissa is wavelength, and the ordinate is ECL intensity.

FIG. 17 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Example 7 is 10,000 pg/mL and the concentration of wild type P₅₃ prepared in Example 7 is 10,000 μM; the abscissa is wavelength, and the ordinate is ECL intensity.

FIG. 18 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Example 8 is 50,000 pg/mL and the concentration of wild type P₅₃ prepared in Example 8 is 50,000 μM; the abscissa is wavelength, and the ordinate is ECL intensity.

FIG. 19 is an ECL spectrogram of the Au NCs prepared in Experiment Example 1 when driven by cyclic voltammetry in 10 mM pH 7.4 Hepes containing 10 mM hydrazine hydrate; the potential window is 0-1.6 V, and the scanning speed is 50 mV/s; the abscissa is potential, and the ordinate is ECL intensity.

FIG. 20 is an ECL spectrogram of the CIS@ZnS NCs prepared in Experiment Example 1 when driven by cyclic voltammetry in 10 mM pH 7.4 Hepes containing 10 mM hydrazine hydrate; the potential window is 0-1.6 V, and the scanning speed is 50 mV/s; the abscissa is potential, and the ordinate is ECL intensity.

FIG. 21 is an ECL spectrogram of the Au NCs and CIS@ZnS NCs prepared in Experiment Example 1 when driven by cyclic voltammetry in 10 mM pH 7.4 Hepes containing 10 mM hydrazine hydrate; the potential window is 0-1.6 V, and the scanning speed is 50 mV/s; the abscissa is potential, and the ordinate is ECL intensity.

FIG. 22 is an ECL spectrogram of the Ab₂|Au NCs prepared in Experiment Example 1 when driven by cyclic voltammetry in 10 mM pH 7.4 Hepes containing 10 mM hydrazine hydrate; the potential window is 0-1.6 V, and the scanning speed is 50 mV/s; the abscissa is potential, and the ordinate is ECL intensity.

FIG. 23 is an ECL spectrogram of the Pₚ₅₃|CIS@ZnS NCs prepared in Experiment Example 1 when driven by cyclic voltammetry in 10 mM pH 7.4 Hepes containing 10 mM hydrazine hydrate; the potential window is 0-1.6 V, and the scanning speed is 50 mV/s; the abscissa is potential, and the ordinate is ECL intensity.

FIG. 24 is an ECL spectrogram of the ECL immunosensor with the Au NCs as labels when the concentration of the CEA prepared in Experiment Example 2 is 0.3 pg/mL; the abscissa is wavelength, and the ordinate is ECL intensity.

FIG. 25 is an ECL spectrogram of the ECL immunosensor with the Au NCs as labels when the concentration of the CEA prepared in Experiment Example 2 is 1 pg/mL; the abscissa is wavelength, and the ordinate is ECL intensity.

FIG. 26 is an ECL spectrogram of the ECL immunosensor with the Au NCs as labels when the concentration of the CEA prepared in Experiment Example 2 is 5 pg/mL; the abscissa is wavelength, and the ordinate is ECL intensity.

FIG. 27 is an ECL spectrogram of the ECL immunosensor with the Au NCs as labels when the concentration of the CEA prepared in Experiment Example 2 is 50 pg/mL; the abscissa is wavelength, and the ordinate is ECL intensity.

FIG. 28 is an ECL spectrogram of the ECL immunosensor with the Au NCs as labels when the concentration of the CEA prepared in Experiment Example 2 is 500 pg/mL; the abscissa is wavelength, and the ordinate is ECL intensity.

FIG. 29 is an ECL spectrogram of the ECL immunosensor with the Au NCs as labels when the concentration of the CEA prepared in Experiment Example 2 is 3,000 pg/mL; the abscissa is wavelength, and the ordinate is ECL intensity.

FIG. 30 is an ECL spectrogram of the ECL immunosensor with the Au NCs as labels when the concentration of the CEA prepared in Experiment Example 2 is 10,000 pg/mL; the abscissa is wavelength, and the ordinate is ECL intensity.

FIG. 31 is an ECL spectrogram of the ECL immunosensor with the Au NCs as labels when the concentration of the CEA prepared in Experiment Example 2 is 50,000 pg/mL; the abscissa is wavelength, and the ordinate is ECL intensity.

FIG. 32 is a working curve of the immunosensor with the Au NCs as labels when the concentrations of CEA prepared in Experiment Example 2 are different; the abscissa is the antigen concentration of a substance to be tested, and the ordinate is ECL intensity.

FIG. 33 is an ECL spectrogram of the ECL immunosensor with the Au NCs as labels when the concentration of the CEA prepared in Experiment Example 2 is 0; the abscissa is wavelength, and the ordinate is ECL intensity.

FIG. 34 is an ECL spectrogram of the ECL sensor for nucleic acid testing with the CIS@ZnS NCs as labels when the concentration of the wild type P₅₃ prepared in Experiment Example 3 is 0.3 μM; the abscissa is wavelength, and the ordinate is ECL intensity.

FIG. 35 is an ECL spectrogram of the ECL sensor for nucleic acid testing with the CIS@ZnS NCs as labels when the concentration of the wild type P₅₃ prepared in Experiment Example 3 is 1 μM; the abscissa is wavelength, and the ordinate is ECL intensity.

FIG. 36 is an ECL spectrogram of the ECL sensor for nucleic acid testing with the CIS@ZnS NCs as labels when the concentration of the wild type P₅₃ prepared in Experiment Example 3 is 5 μM; the abscissa is wavelength, and the ordinate is ECL intensity.

FIG. 37 is an ECL spectrogram of the ECL sensor for nucleic acid testing with the CIS@ZnS NCs as labels when the concentration of the wild type P₅₃ prepared in Experiment Example 3 is 50 μM; the abscissa is wavelength, and the ordinate is ECL intensity.

FIG. 38 is an ECL spectrogram of the ECL sensor for nucleic acid testing with the CIS@ZnS NCs as labels when the concentration of the wild type P₅₃ prepared in Experiment Example 3 is 500 μM; the abscissa is wavelength, and the ordinate is ECL intensity.

FIG. 39 is an ECL spectrogram of the ECL sensor for nucleic acid testing with the CIS@ZnS NCs as labels when the concentration of the wild type P₅₃ prepared in Experiment Example 3 is 3,000 μM; the abscissa is wavelength, and the ordinate is ECL intensity.

FIG. 40 is an ECL spectrogram of the ECL sensor for nucleic acid testing with the CIS@ZnS NCs as labels when the concentration of the wild type P₅₃ prepared in Experiment Example 3 is 10,000 μM; the abscissa is wavelength, and the ordinate is ECL intensity.

FIG. 41 is an ECL spectrogram of the ECL sensor for nucleic acid testing with the CIS@ZnS NCs as labels when the concentration of the wild type P₅₃ prepared in Experiment Example 3 is 50,000 μM; the abscissa is wavelength, and the ordinate is ECL intensity.

FIG. 42 is a working curve of the ECL sensor for nucleic acid testing and the CIS@ZnS NCs as labels when the concentrations of wild type P₅₃ prepared in Experiment Example 3 are different; the abscissa is the target DNA concentration, and the ordinate is ECL intensity.

FIG. 43 is an ECL spectrogram of the ECL sensor for nucleic acid testing with the CIS@ZnS NCs as labels when the concentration of the wild type P₅₃ prepared in Experiment Example 3 is 0; the abscissa is wavelength, and the ordinate is ECL intensity.

FIG. 44 is an ECL response specificity diagram of Au|MPA−Cₚ₅₃^{Ab1}<Tₚ₅₃^{Ag}>Pₚ₅₃^{Ab2}|CIS_{@ZnSNCs}^{Au NCs} in Experiment Example 1; the abscissa is the type of a substance to be tested, and the ordinate is ECL intensity.

FIG. 45 is an ECL spectrogram of Au|MPA−Cₚ₅₃^{Ab1}<Tₚ₅₃^{Ag}>Pₚ₅₃^{Ab2}|CIS_{@ZnSNCs}^{Au NCs} when the concentration of CEA prepared in Comparative Example 1 is 0 and the concentration of wild type P₅₃ is 0; the abscissa is wavelength, and the ordinate is ECL intensity.

### DETAILED DESCRIPTION OF THE PREFERRED EMBODIMENTS

The present invention will be further described below through the embodiments, but the present invention is not limited thereinto.

ECL spectrum acquisition in the examples is performed with a testing system capable of accurately acquiring ECL spectrum information in ZL201620300698.3. For a mode of ECL spectrum acquisition, refer to the spectrum acquisition method of ECL immunoassay established in ZL201610237580.5, the potential window used is 0˜1.6 V, the scanning speed is 50 mV/s, the initial potential is 0 V, and the initial scanning direction is positive.

All the raw materials used in the examples are commercially available products, unless otherwise specified.

Target DNA
(Tₚ₅₃, CGT GGA GCT ACA GTG GTA AAG CAG GTG AAG AAA
TGC AGT (SEQ ID NO.: 01)) in examples
Captured DNA
(Cₚ₅₃, 5′-NH₂-(CH₂)₆-GTA ACT GCA TTT CTT CAC CTG
(SEQ ID NO.: 02))
Probe DNA
(Pₚ₅₃, CAC TGT AGC TCC ACG ACC-(CH₂)₆-NH₂-3′ (SEQ
ID NO.: 03))
Single-base mismatched target DNA1
(M1, CGT GGA GTT ACA GTG GTA AAG CAG GTG AAG AAA
TGC AGT (SEQ ID NO.: 04))
Double-base mismatched target DNA2
(M2, CGT GGA GTT ACA GTG GTA AAG CAG GTG TAG AAA
TGC AGT (SEQ ID NO.: 05)
Three-base mismatched target DNA3
(M3, CGT GGA GTT ACA GCG GTA AAG CAG GTG TAG AAA
TGC AGT (SEQ ID NO.: 06))

### EXAMPLE 1

Preparation of water-soluble Au NCs:
(1)35.5 μL of 100 mg/mL HAuCl₄·3H₂O was taken, and 2.5 mL of deionized water was added;
(2)50 μL of mercaptopropionic acid was added to the mixture in step (1), and the mixture was stirred for 15 min;
(3)430 μL of 1 M sodium hydroxide was added to the mixture in step (2), and the pH was adjusted to 8.5;
(4)0.5 mL of 0.1 M zinc acetate was added to the mixture in step (3), the mixture was stirred at room temperature for a reaction for 6 h, the reaction product was washed and purified with isopropanol after the reaction was completed and dissolved in deionized water to obtain a monodisperse solution of water-soluble Au NCs.

Product Characterization:

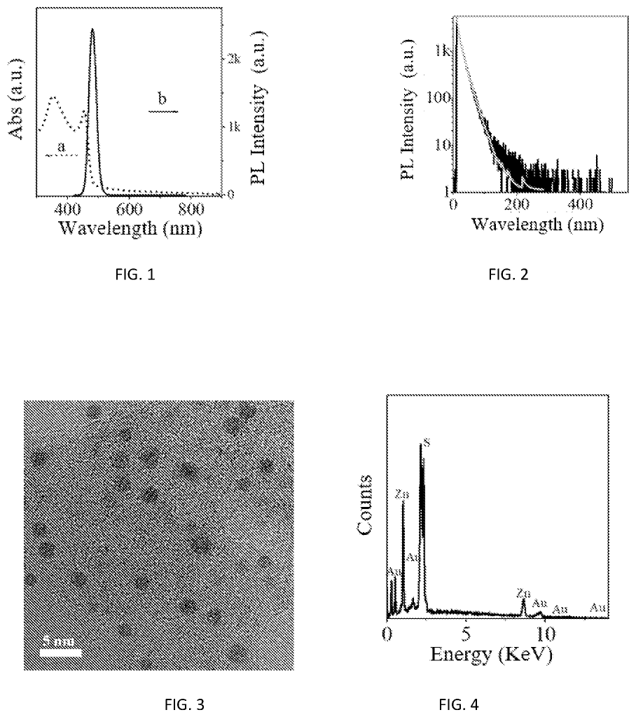

An ultraviolet absorption diagram of the Au NCs obtained in this example is shown in FIG. 1; the UV absorption characteristic peaks are at 355 nm and 450 nm.

A fluorescence emission spectrogram of the Au NCs is shown in FIG. 1; a fluorescence emission characteristic peak is at 485 nm, and a half-peak width is 25 nm.

A fluorescence lifetime diagram of the Au NCs is shown in FIG. 2; the fluorescence lifetime of the Au NCs is 31 ns.

A TEM image of the Au NCs is shown in FIG. 3; the Au NCs are spherical, with an average size of 4.4 nm.

An element distribution diagram of the Au NCs is shown in FIG. 4; the Au NCs are composed of Au, S, Zn and other elements.

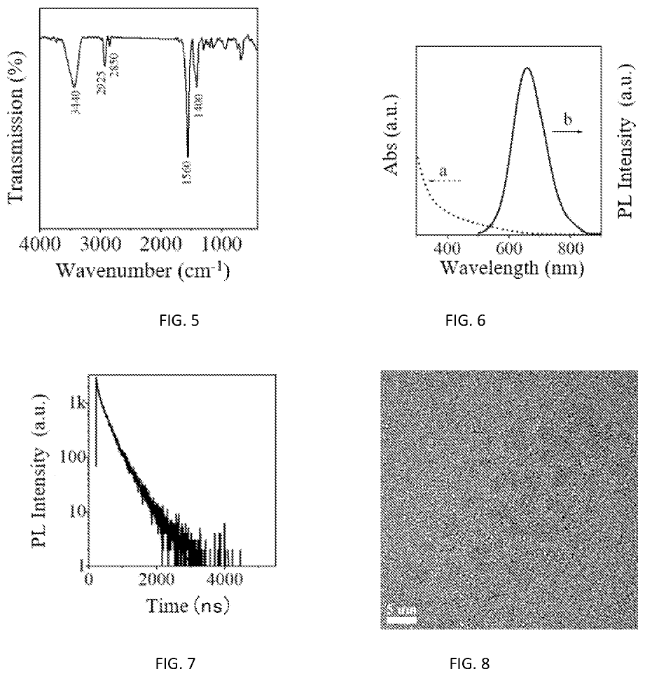

An infrared spectrum of the Au NCs is shown in FIG. 5; the surface of the Au NCs is rich in carboxylic acid groups.

Preparation of water-soluble CIS@ZnS NCs quantum dots:
(1)0.0022 g of captopril, 0.01 g of NaOH, 0.0471 g of sodium citrate, 0.0017 g of CuCl₂·2H₂O, 0.0117 g of InCl₃·4H₂O and 0.0048 g of Na₂S were dissolved in 20 mL of deionized water successively under a constant stirring condition, the mixture was heated to 95° C., and held for 45 min;
(2)0.177 g of Zn(CH₃COO)₂ and 0.061 g of thiourea were added to the mixture in step (1);
(3)isopropanol was added to the mixture in step (2) for washing and purification to obtain CIS@ZnS NCs.

Product Characterization:

An ultraviolet absorption diagram of the CIS@ZnS NCs obtained in this example is shown in FIG. 6; the CIS@ZnS NCs have no obvious UV absorption peaks.

A fluorescence emission spectrogram of the CIS@ZnS NCs is shown in FIG. 6; a fluorescence emission characteristic peak is at 660 nm, and a half-peak width is 120 nm.

A fluorescence lifetime diagram of the CIS@ZnS NCs is shown in FIG. 7; the fluorescence lifetime of the CIS@ZnS NCs is 330 ns.

A TEM image of the Au NCs is shown in FIG. 8; the CIS@ZnS NCs are spherical, with an average size of 4.5 nm.

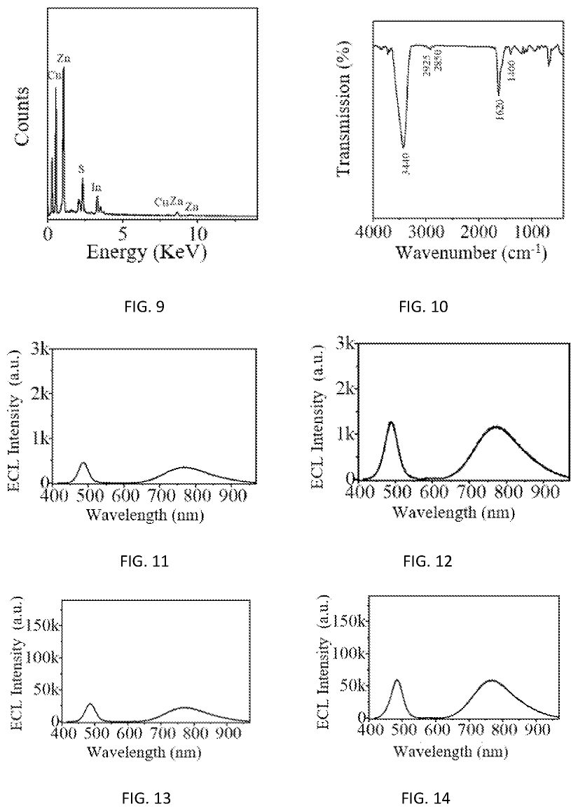

An element distribution diagram of the CIS@ZnS NCs is shown in FIG. 9; the CIS@ZnS NCs are composed of Cu, In, S, Zn and other elements.

An infrared spectrum of the CIS@ZnS NCs is shown in FIG. 10; the surface of the CIS@ZnS NCs is rich in carboxylic acid groups.

Preparation of a double-labeled Au electrode:
(1)a cleaned Au electrode was soaked in 10 mM mercaptopropionic acid overnight, and MPA was bonded to the surface of the electrode through an Au—S bond;
(2)10 μL of 10 mg/mL EDC and 10 mg/mL NHS were added dropwise to the surface of the modified electrode obtained in step (1), the mixture was activated for 30 min, the electrode was cleaned, and unreacted EDC and NHS were removed;
(3)10 μL of 10 μg/mL aqueous solution of CEA-Ab₁ and 10 μL of 10 μM aqueous solution of Cₚ₅₃ were incubated for 2-4 h, the mixture was added to unreacted active sites on the BSA closed electrode, and the electrode was cleaned to obtain a double-labeled Au electrode: Au|MPA−Cₚ₅₃^{Ab1}

Preparation of Ab₂|Au NCs:

purified Au NCs were dissolved in 1 mL of 0.1 M pH 6.0 PBS containing 10 mg/mL EDC and 10 mg/mL NHS, the mixture was activated for 30 min, centrifugal purification was performed, and the mixture was dispersed in 1 mL of pH 7.4 0.1 M PBS to obtain activated Au NCs; 10 μL of 10 μg/mL aqueous solution of CEA-Ab₂ was added, the mixture was incubated at a constant temperature of 37° C. for 3-5 h, 20 μL of BSA was added, the mixture was sealed for 30 min and centrifuged, and sediments were collected to obtain Ab₂|Au NCs.

Preparation of Pₚ₅₃|CIS@ZnS NCs:
purified CIS@ZnS NCs were dissolved in 1 mL of 0.1 M pH 6.0 PBS containing 10 mg/mL EDC and 10 mg/mL NHS, the mixture was activated for 30 min, centrifugal purification was performed, and the mixture was dispersed in 1 mL of pH 7.4 0.1 M PBS to obtain activated CIS@ZnS NCs; 10 μL of 10 μM aqueous solution of Pₚ₅₃ was added, the mixture was incubated at a constant temperature of 37° C. for 3-5 h, an amino group at one end of the probe DNA and a carboxyl group on the surface of CIS@ZnS NCs were connected through an amidation reaction, 20 μL of BSA was added, the mixture was sealed for 30 min and centrifuged, and sediments were collected to obtain Pₚ₅₃|CIS@ZnS NCs.

Establishment of the ECL Multicomponent Analysis Sensor for Synchronous Implementation of Immunoassay and Nucleic Acid Testing:
a. a cleaned Au electrode was soaked in 10 mM mercaptopropionic acid overnight, and MPA was bonded to the surface of the electrode through an Au—S bond;
b. 10 μL of 10 mg/mL EDC and 10 mg/mL NHS were added dropwise to the surface of the modified electrode obtained in a, the mixture was activated for 30 min, the electrode was cleaned, and unreacted EDC and NHS were removed;
c. 10 μL of 10 μg/mL aqueous solution of CEA-Ab₁ and 10 μL of 10 μM aqueous solution of Cₚ₅₃ were mixed and added to the surface of the activated electrode obtained in step b, incubated for 3 h and added to unreacted active sites on a BSA closed electrode, and the electrode was cleaned to obtain a double-labeled Au electrode;
d. purified Au NCs were dissolved in 1 mL of 0.1 M pH 6.0 PBS containing 10 mg/mL EDC and 10 mg/mL NHS, the mixture was activated for 30 min, centrifugal purification was performed, and the mixture was dispersed in 1 mL of pH 7.4 0.1 M PBS to obtain activated Au NCs; 10 μL of 10 μg/mL aqueous solution of CEA-Ab₂ was added, the mixture was incubated at a constant temperature of 37° C. for 3-5 h, 20 μL of BSA was added, the mixture was sealed for 30 min and centrifuged, and sediments were collected to obtain Ab₂|Au NCs;
e. purified CIS@ZnS NCs were dissolved in 1 mL of 0.1 M pH 6.0 PBS containing 10 mg/mL EDC and 10 mg/mL NHS, the mixture was activated for 30 min, centrifugal purification was performed, and the mixture was dispersed in 1 mL of pH 7.4 0.1 M PBS to obtain activated CIS@ZnS NCs; 10 μL of 10 μM aqueous solution of probe DNA was added, the mixture was incubated at a constant temperature of 37° C. for 3-5 h, 20 μL of BSA was added, the mixture was sealed for 30 min and centrifuged, and sediments were collected to obtain Pₚ₅₃|CIS@ZnS NCs;
f. CEA-Ag and Tₚ₅₃ were added dropwise to the surface of the double-labeled Au electrode, the mixture was incubated at room temperature for 90 min, the electrode was cleaned, and the Ab₂|Au NCs and Pₚ₅₃|CIS@ZnS NCs were added dropwise to the surface of the electrode and incubated for 1 h; Ab₂|Au NCs and Pₚ₅₃|CIS@ZnS NCs were grafted and fixed to the surface of the working electrode in a form of immune complex formation to obtain an ECL multicomponent analysis sensor capable of enabling synchronous implementation of immunoassay and nucleic acid testing.

Immunoassay-Nucleic Acid Testing Synchronous ECL Multicomponent Analysis Method:
I: 10 μL of 0.3 pg/mL CEA-Ag and 10 μL of 0.5 μM Tₚ₅₃ were mixed and added to the surface of a double-labeled Au electrode dropwise, the mixture was incubated at room temperature for 90 min, the electrode was cleaned, and the Ab₂|Au NCs and Pₚ₅₃|CIS@ZnS NCs were added to the surface of the electrode dropwise and incubated for 1 h; the Ab₂|Au NCs and Pₚ₅₃|CIS@ZnS NCs were grafted and fixed to the surface of a working electrode to obtain an ECL sensor for synchronous implementation of immunoassay and nucleic acid testing, and ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the ECL sensor could generate dual-emission ECL signals with maximum emission wavelengths at 485 nm and 775 nm in 10 mM pH 7.4 Hepes buffer solution containing hydrazine hydrate (the ECL spectrum is shown in FIG. 11);
II: all photons in the whole process of ECL were collected by means of exposure imaging, and a total spectrum was obtained by means of radiation of all the photons based on dispersive ECL; a working curve of CEA testing was drawn according to a relationship between the maximum radiation intensity at the maximum radiation wavelength of 485 nm on a spectral curve and the concentration of a standard antigen solution; a working curve of Tₚ₅₃ testing was drawn according to a relationship between the maximum radiation intensity at the maximum radiation wavelength of 775 nm on the spectral curve and the concentration of standard target DNA Tₚ₅₃;
III: an ECL multicomponent analysis sensor was established according to the method for establishing an ECL sensor for synchronous implementation of immunoassay and nucleic acid testing using a target DNA to be tested and CEA-Ag to be tested; ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the concentrations of the antigen and target DNA in the sample solution to be tested were synchronously tested according to a light intensity signal and a working curve at the maximum radiation wavelength on the obtained ECL spectral curve.

### EXAMPLE 2

The differences from the immunoassay-nucleic acid testing synchronous ECL multicomponent analysis method of Example 1 were as follows:

In step I, 10 μL of 1 pg/mL CEA-Ag and 10 μL of 1 μM Tₚ₅₃ were added to the surface of a double-labeled Au electrode dropwise, the mixture was incubated at room temperature for 90 min, the electrode was cleaned, and the Ab₂|Au NCs and Pₚ₅₃|CIS@ZnS NCs were added to the surface of the electrode dropwise and incubated for 1 h; the Ab₂|Au NCs and Pₚ₅₃|CIS@ZnS NCs were grafted and fixed to the surface of a working electrode to obtain an ECL sensor for synchronous implementation of immunoassay- and nucleic acid testing, and ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the ECL sensor could generate dual-emission ECL signals with maximum emission wavelengths at 485 nm and 775 nm in 10 mM pH 7.4 Hepes buffer solution containing hydrazine hydrate (the ECL spectrum is shown in FIG. 12).

### EXAMPLE 3

The differences from the immunoassay-nucleic acid testing synchronous ECL multicomponent analysis method of Example 1 were as follows:

In step I, 10 μL of 5 pg/mL CEA-Ag and 10 μL of 5 μM Tₚ₅₃ were added to the surface of a double-labeled Au electrode dropwise, the mixture was incubated at room temperature for 90 min, the electrode was cleaned, and the Ab₂|Au NCs and Pₚ₅₃|CIS@ZnS NCs were added to the surface of the electrode dropwise and incubated for 1 h; the Ab₂|Au NCs and Pₚ₅₃|CIS@ZnS NCs were grafted and fixed to the surface of a working electrode to obtain an ECL sensor for synchronous implementation of immunoassay and nucleic acid testing, and ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the ECL sensor could generate dual-emission ECL signals with maximum emission wavelengths at 485 nm and 775 nm in 10 mM pH 7.4 Hepes buffer solution containing hydrazine hydrate (the ECL spectrum is shown in FIG. 13).

### EXAMPLE 4

The differences from the immunoassay-nucleic acid testing synchronous ECL multicomponent analysis method of Example 1 are as follows:

In step I, 10 μL of 50 pg/mL CEA-Ag and 10 μL of 50 μM Tₚ₅₃ were added to the surface of a double-labeled Au electrode dropwise, the mixture was incubated at room temperature for 90 min, the electrode was cleaned, and the Ab₂|Au NCs and Pₚ₅₃|CIS@ZnS NCs were added to the surface of the electrode dropwise and incubated for 1 h; the Ab₂|Au NCs and Pₚ₅₃|CIS@ZnS NCs were grafted and fixed to the surface of a working electrode to obtain an ECL sensor for synchronous implementation of immunoassay and nucleic acid testing, and ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the ECL sensor could generate dual-emission ECL signals with maximum emission wavelengths at 485 nm and 775 nm in 10 mM pH 7.4 Hepes buffer solution containing hydrazine hydrate (the ECL spectrum is shown in FIG. 14).

### EXAMPLE 5

The differences from the immunoassay-nucleic acid testing synchronous ECL multicomponent analysis method of Example 1 were as follows:

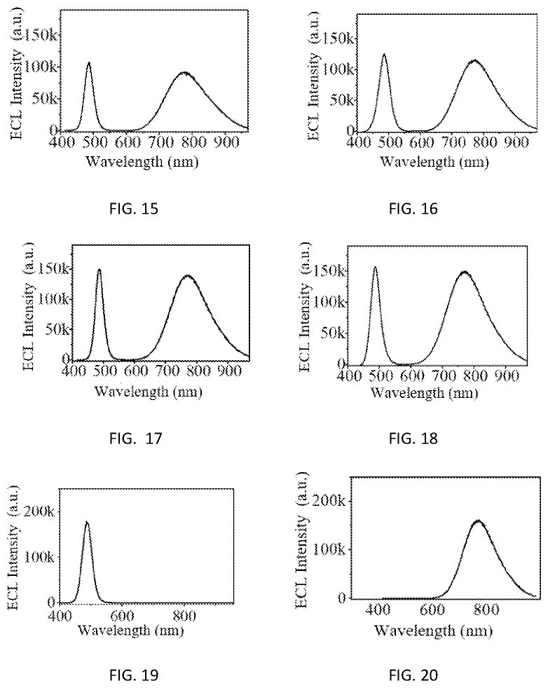

In step I, 10 μL of 500 pg/mL CEA-Ag and 10 μL of 500 μM Tₚ₅₃ were added to the surface of a double-labeled Au electrode dropwise, the mixture was incubated at room temperature for 90 min, the electrode was cleaned, and the Ab₂|Au NCs and Pₚ₅₃|CIS@ZnS NCs were added to the surface of the electrode dropwise and incubated for 1 h; the Ab₂|Au NCs and Pₚ₅₃|CIS@ZnS NCs were grafted and fixed to the surface of a working electrode to obtain an ECL sensor for synchronous implementation of immunoassay and nucleic acid testing, and ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the ECL sensor could generate dual-emission ECL signals with maximum emission wavelengths at 485 nm and 775 nm in 10 mM pH 7.4 Hepes buffer solution containing hydrazine hydrate (the ECL spectrum is shown in FIG. 15).

### EXAMPLE 6

The differences from the immunoassay-nucleic acid testing synchronous ECL multicomponent analysis method of Example 1 were as follows:

In step I, 10 μL of 3,000 pg/mL CEA-Ag and 10 μL of 3,000 μM Tₚ₅₃ were added to the surface of a double-labeled Au electrode dropwise, the mixture was incubated at room temperature for 90 min, the electrode was cleaned, and the Ab₂|Au NCs and Pₚ₅₃|CIS@ZnS NCs were added to the surface of the electrode dropwise and incubated for 1 h; the Ab₂|Au NCs and Pₚ₅₃|CIS@ZnS NCs were grafted and fixed to the surface of a working electrode to obtain an ECL sensor for synchronous implementation of immunoassay and nucleic acid testing, and ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the ECL sensor could generate dual-emission ECL signals with maximum emission wavelengths at 485 nm and 775 nm in 10 mM pH 7.4 Hepes buffer solution containing hydrazine hydrate (the ECL spectrum is shown in FIG. 16).

### EXAMPLE 7

The differences from the immunoassay-nucleic acid testing synchronous ECL multicomponent analysis method of Example 1 were as follows:

In step I, 10 μL of 10,000 pg/mL CEA-Ag and 10 μL of 10,000 μM Tₚ₅₃ were added to the surface of a double-labeled Au electrode dropwise, the mixture was incubated at room temperature for 90 min, the electrode was cleaned, and the Ab₂|Au NCs and Pₚ₅₃|CIS@ZnS NCs were added to the surface of the electrode dropwise and incubated for 1 h; the Ab₂|Au NCs and Pₚ₅₃|CIS@ZnS NCs were grafted and fixed to the surface of a working electrode to obtain an ECL sensor for synchronous implementation of immunoassay and nucleic acid testing, and ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the ECL sensor could generate dual-emission ECL signals with maximum emission wavelengths at 485 nm and 775 nm in 10 mM pH 7.4 Hepes buffer solution containing hydrazine hydrate (the ECL spectrum is shown in FIG. 17).

### EXAMPLE 8

The differences from the immunoassay-nucleic acid testing synchronous ECL multicomponent analysis method of Example 1 were as follows:

In step I, 10 μL of 50,000 pg/mL CEA-Ag and 10 μL of 50,000 μM Tₚ₅₃ were added to the surface of a double-labeled Au electrode dropwise, the mixture was incubated at room temperature for 90 min, the electrode was cleaned, and the Ab₂|Au NCs and Pₚ₅₃|CIS@ZnS NCs were added to the surface of the electrode dropwise and incubated for 1 h; the Ab₂|Au NCs and Pₚ₅₃|CIS@ZnS NCs were grafted and fixed to the surface of a working electrode to obtain an ECL sensor for synchronous implementation of immunoassay and nucleic acid testing, and ECL was produced by taking the obtained sensor electrode as a working electrode, a platinum electrode as a counter electrode, and an Ag/AgCl electrode as a reference electrode when cyclic voltammetry was used for driving in a Hepes buffer solution containing 10 mM hydrazine hydrate; the ECL sensor could generate dual-emission ECL signals with maximum emission wavelengths at 485 nm and 775 nm in 10 mM pH 7.4 Hepes buffer solution containing hydrazine hydrate (the ECL spectrum is shown in FIG. 18).

Experiment Example 1 Verification of ECL system:
1. The Au NCs prepared in Example 1 were diluted into 0.15 mg/mL monodisperse solution.

An Au electrode was used as a working electrode, a platinum filament was used as a counter electrode, an Ag/AgCl electrode was used as a reference electrode, 10 mM pH 7.4 Hepes was used as a buffer solution, 10 mM hydrazine hydrate was used as a coreactant, and 0.15 mg/mL Au NCs monodisperse solution was used as a luminescent reagent.

Cyclic voltammetry was used for driving, a potential window was 0-1.6 V, a scanning speed was 50 mV/s, an initial potential was 0 V, and an initial scanning direction was positive. The Au NCs could generate a highly monochromatic ECL signal with a maximum emission wavelength at 485 nm in a 10 mM pH 7.4 Hepes buffer solution containing hydrazine hydrate, with a half-peak width of 36 nm (the ECL spectrum is shown in FIG. 19).

2. The CIS@ZnS NCs prepared in Example 1 were diluted into 1.0 mg/mL monodisperse solution.

An Au electrode was used as a working electrode, a platinum filament was used as a counter electrode, an Ag/AgCl electrode was used as a reference electrode, 10 mM pH 7.4 Hepes was used as a buffer solution, 10 mM hydrazine hydrate was used as a coreactant, and 1.0 mg/mL CIS@ZnS NCs monodisperse solution was used as a luminescent reagent.

Cyclic voltammetry was used for driving, a potential window was 0-1.6 V, a scanning speed was 50 mV/s, an initial potential was 0 V, and an initial scanning direction was positive. The CIS@ZnS NCs could generate an ECL signal with a maximum emission wavelength at 775 nm in a 10 mM pH 7.4 Hepes buffer solution containing hydrazine hydrate (the ECL spectrum is shown in FIG. 20).

3. An Au electrode was used as a working electrode, a platinum filament was used as a counter electrode, an Ag/AgCl electrode was used as a reference electrode, 10 mM pH 7.4 Hepes was used as a buffer solution, 10 mM hydrazine hydrate was used as a coreactant, and a liquid mixture formed after mixing 0.15 mg/mL Au NCs prepared in Example 1 and 1.0 mg/mL CIS@ZnS NCs prepared in Example 1 was used as a luminescent reagent.

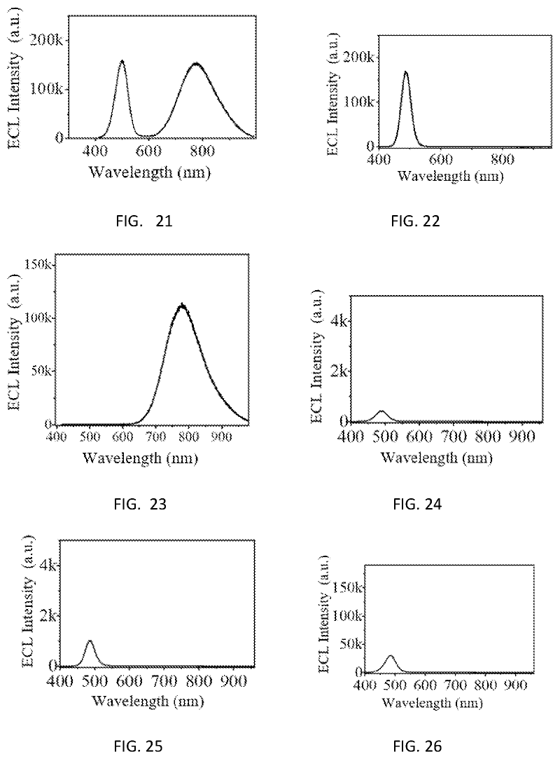

Cyclic voltammetry was used for driving, a potential window was 0-1.6 V, a scanning speed was 50 mV/s, an initial potential was 0 V, and an initial scanning direction was positive. The Au NCs and CIS@ZnS NCs could generate a dual-band ECL signal with a maximum emission wavelength at 485 nm and 775 nm in a 10 mM pH 7.4 Hepes buffer solution containing hydrazine hydrate (the ECL spectrum is shown in FIG. 21).

4. An Au electrode was used as a working electrode, a platinum filament was used as a counter electrode, an Ag/AgCl electrode was used as a reference electrode, 10 mM pH 7.4 Hepes was used as a buffer solution, 10 mM hydrazine hydrate was used as a coreactant, and 0.15 mg/mL aqueous solution of Ab₂|Au NCs obtained in step d in Example 1 was used as a luminescent reagent.

Cyclic voltammetry was used for driving, a potential window was 0-1.6 V, a scanning speed was 50 mV/s, an initial potential was 0 V, and an initial scanning direction was positive. The Ab₂|Au NCs could generate a highly monochromatic ECL signal with a maximum emission wavelength at 485 nm in a 10 mM pH 7.4 Hepes buffer solution containing hydrazine hydrate, with a half-peak width of 36 nm (the ECL spectrum is shown in FIG. 22). Although the ECL intensity was lower than that of the Au NCs, which was attributed to a steric effect of antibodies to proteins, it proved that the secondary antibody had been grafted onto the Au NCs successfully.

5. An Au electrode was used as a working electrode, a platinum filament was used as a counter electrode, an Ag/AgCl electrode was used as a reference electrode, 10 mM pH 7.4 Hepes was used as a buffer solution, 10 mM hydrazine hydrate was used as a coreactant, and 1.0 mg/mL aqueous solution of Pₚ₅₃|CIS@ZnS NCs obtained in step e in Example 1 was used as a luminescent reagent.

Cyclic voltammetry was used for driving, a potential window was 0-1.6 V, a scanning speed was 50 mV/s, an initial potential was 0 V, and an initial scanning direction was positive. The Pₚ₅₃|CIS@ZnS NCs could generate an ECL signal with a maximum emission wavelength at 775 nm in a 10 mM pH 7.4 Hepes buffer solution containing hydrazine hydrate, with an ECL intensity lower than that of CIS@ZnS NCs (the ECL spectrum is shown in FIG. 23).

### EXPERIMENT EXAMPLE 2

1. Establishment of an ECL immunosensor with Au NCs as labels:
a cleaned Au electrode was soaked in 10 mM mercaptopropionic acid overnight, 200 μL of MPA was added to react for 10 h, and the MPA was bonded to the surface of the electrode through an Au—S bond;
10 μL of 10 mg/mL EDC and 10 mg/mL NHS were added dropwise to the surface of the modified electrode obtained in a, the mixture was activated for 30 min, the electrode was cleaned with 10 mM pH 7.4 PBS, and unreacted EDC and NHS were removed;
20 μL of 10 mg/mL CEA-Ab₁ was added dropwise to the surface of the activated electrode obtained in b, incubated for 3 h and added to unreacted active sites on a BSA closed electrode, and the electrode was cleaned with 10 mM pH 7.4 PBS;
purified Au NCs were dissolved in a phosphate buffer solution containing EDC and NHS and activated, then CEA-Ab₂ was added, the mixture was incubated at a constant temperature of 37C for 3-5 h, BSA was added, and the mixture was sealed for 30 min to obtain Ab₂|Au NCs;
10 μL of aqueous solution of CEA-Ag was added dropwise to the surface of the electrode treated in c and incubated at room temperature for 90 min, the electrode was cleaned with 10 mM pH 7.4 PBS, and 10 μL of Ab₂|Au NCs was added dropwise to the surface of the electrode and incubated for 1 h; the Ab₂|Au NCs were grafted and fixed to the surface of the working electrode based on a form of immune complex formation to obtain a highly monochromatic ECL immunosensor.
   (1) An electrochemical immunosensor was prepared according to the steps described in Example 1, 10 μL of Ag labeled carcinoembryonic antigen (CEA-Ag) was added dropwise to the surface of the electrode treated in c, with antigen concentrations of: 0.3 pg/mL, 1 pg/mL, 5 pg/mL, 50 pg/mL, 500 pg/mL, 3,000 pg/mL, 10,000 pg/mL and 50,000 pg/mL respectively to obtain a highly monochromatic ECL immunosensor with carcinoembryonic antigens of different concentrations;
   (1)the ECL immunosensor was used as a working electrode, a platinum filament was used as a counter electrode, an Ag/AgCl electrode was used as a reference electrode, 10 mM pH 7.4 Hepes was used as a buffer solution, and 10 mM hydrazine hydrate was used as a coreactant. Cyclic voltammetry was used for driving, a potential window was 0-1.6 V, a scanning speed was 50 mV/s, an initial potential was 0 V, an initial scanning direction was positive, and an ECL spectrum obtained with antigens of different concentrations is shown in FIGS. 24-31;
   (2)all photons in the whole process of ECL were collected by means of exposure imaging, and a total spectrum was obtained by means of radiation of all the photons based on dispersive ECL; a working curve was drawn according to a relationship between light intensity at the maximum radiation wavelength on the spectral curve and the concentration of a standard antigen solution; the working curve of the ECL immunosensor for antigens is shown in FIG. 24. An ECL signal was gradually enhanced as the antigen concentration increased.

When the concentration of the CEA was 0.3 pg/mL, an ECL spectrogram of the ECL immunosensor with Au NCs as labels is shown in FIG. 24. The ECL immunosensor could generate a highly monochromatic ECL signal with the maximum emission wavelength at 485 nm in 10 mM pH 7.4 Hepes buffer solution containing hydrazine hydrate, with a half-peak width of 36 nm; compared with the ECL spectrogram of the ECL sensor for synchronous implementation of immunoassay and nucleic acid testing in Example 1, the ECL signals at 485 nm were basically the same, and an immunoassay process and a nucleic acid testing process did not affect each other under this condition.

When the concentration of the CEA was 1 pg/mL, an ECL spectrogram of the ECL immunosensor with Au NCs as labels is shown in FIG. 25. The ECL immunosensor could generate a highly monochromatic ECL signal with the maximum emission wavelength at 485 nm in 10 mM pH 7.4 Hepes buffer solution containing hydrazine hydrate, with a half-peak width of 36 nm. Compared with the ECL spectrogram of the ECL sensor for synchronous implementation of immunoassay and nucleic acid testing in Example 2, the ECL signals at 485 nm were basically the same, and an immunoassay process and a nucleic acid testing process did not affect each other under this condition.

When the concentration of the CEA was 5 pg/mL, an ECL spectrogram of the ECL immunosensor with Au NCs as labels is shown in FIG. 26. The ECL immunosensor could generate a highly monochromatic ECL signal with the maximum emission wavelength at 485 nm in 10 mM pH 7.4 Hepes buffer solution containing hydrazine hydrate, with a half-peak width of 36 nm. Compared with the ECL spectrogram of the ECL sensor for synchronous implementation of immunoassay and nucleic acid testing in Example 3, the ECL signals at 485 nm were basically the same, and an immunoassay process and a nucleic acid testing process did not affect each other under this condition.

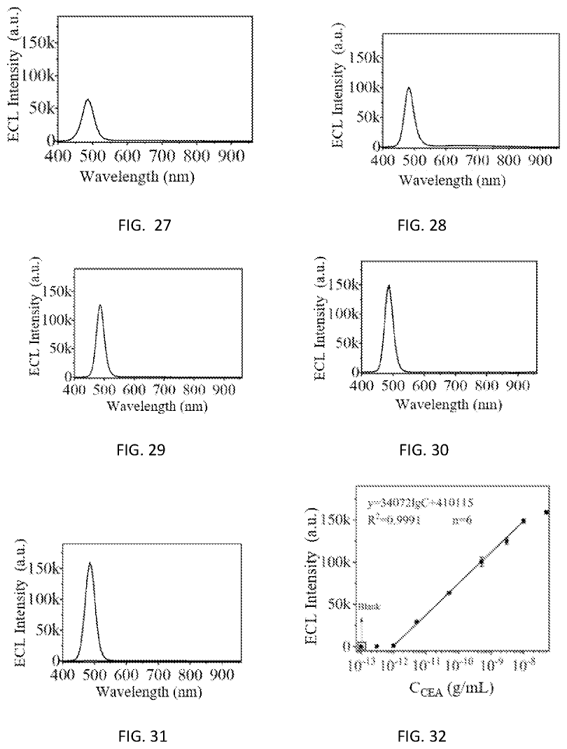

When the concentration of the CEA was 50 pg/mL, an ECL spectrogram of the ECL immunosensor with Au NCs as labels is shown in FIG. 27. The ECL immunosensor could generate a highly monochromatic ECL signal with the maximum emission wavelength at 485 nm in 10 mM pH 7.4 Hepes buffer solution containing hydrazine hydrate, with a half-peak width of 36 nm. Compared with the ECL spectrogram of the ECL sensor for synchronous implementation of immunoassay and nucleic acid testing in Example 4, the ECL signals at 485 nm were basically the same, and an immunoassay process and a nucleic acid testing process did not affect each other under this condition.

When the concentration of the CEA was 500 pg/mL, an ECL spectrogram of the ECL immunosensor with Au NCs as labels is shown in FIG. 28. The ECL immunosensor could generate a highly monochromatic ECL signal with the maximum emission wavelength at 485 nm in 10 mM pH 7.4 Hepes buffer solution containing hydrazine hydrate, with a half-peak width of 36 nm. Compared with the ECL spectrogram of the ECL sensor for synchronous implementation of immunoassay and nucleic acid testing in Example 5, the ECL signals at 485 nm were basically the same, and an immunoassay process and a nucleic acid testing process did not affect each other under this condition.

When the concentration of the CEA was 3,000 pg/mL, an ECL spectrogram of the ECL immunosensor with Au NCs as labels is shown in FIG. 29. The ECL immunosensor could generate a highly monochromatic ECL signal with the maximum emission wavelength at 485 nm in 10 mM pH 7.4 Hepes buffer solution containing hydrazine hydrate, with a half-peak width of 36 nm. Compared with the ECL spectrogram of the ECL sensor for synchronous implementation of immunoassay and nucleic acid testing in Example 6, the ECL signals at 485 nm were basically the same, and an immunoassay process and a nucleic acid testing process did not affect each other under this condition.

When the concentration of the CEA was 10,000 pg/mL, an ECL spectrogram of the ECL immunosensor with Au NCs as labels is shown in FIG. 30. The ECL immunosensor could generate a highly monochromatic ECL signal with the maximum emission wavelength at 485 nm in 10 mM pH 7.4 Hepes buffer solution containing hydrazine hydrate, with a half-peak width of 36 nm. Compared with the ECL spectrogram of the ECL sensor for synchronous implementation of immunoassay and nucleic acid testing in Example 7, the ECL signals at 485 nm were basically the same, and an immunoassay process and a nucleic acid testing process did not affect each other under this condition.

When the concentration of the CEA was 50,000 pg/mL, an ECL spectrogram of the ECL immunosensor with Au NCs as labels is shown in FIG. 31. The ECL immunosensor could generate a highly monochromatic ECL signal with the maximum emission wavelength at 485 nm in 10 mM pH 7.4 Hepes buffer solution containing hydrazine hydrate, with a half-peak width of 36 nm. Compared with the ECL spectrogram of the ECL sensor for synchronous implementation of immunoassay and nucleic acid testing in Example 8, the ECL signals at 485 nm were basically the same, and an immunoassay process and a nucleic acid testing process did not affect each other under this condition.

The working curve of the ECL immunosensor is shown in FIG. 32. An ECL signal was gradually enhanced as the antigen concentration increased, which proved that the immunosensor had superior performance. However, the light intensities of the working curve of the ECL sensor for synchronous implementation of immunoassay and nucleic acid testing of the present invention under different concentrations were equivalent to that for separate testing. Judging from the ECL curves and spectra, each showed the same degree of decline after labeling, which proved that the immunoassay process and the nucleic acid testing process were basically not affected.

3. Ag (CEA) added to the surface of Au-MPA-Ab₁ dropwise was removed to prepare and obtain an ECL immunosensor Au-MPA-Ab₁<Ag>Ab₂-AuNCs.

Au-|MPA-Ab₁<Ag was used as a working electrode, a platinum filament was used as a counter electrode, an Ag/AgCl electrode was used as a reference electrode, 10 mM pH 7.4 Hepes was used as a buffer solution, and 10 mM hydrazine hydrate was used as a coreactant. Cyclic voltammetry was used for driving, a potential window was 0-1.6 V, a scanning speed was 50 mV/s, an initial potential was 0 V, an initial scanning direction was positive, and an ECL spectrum with different antigen concentrations was obtained.

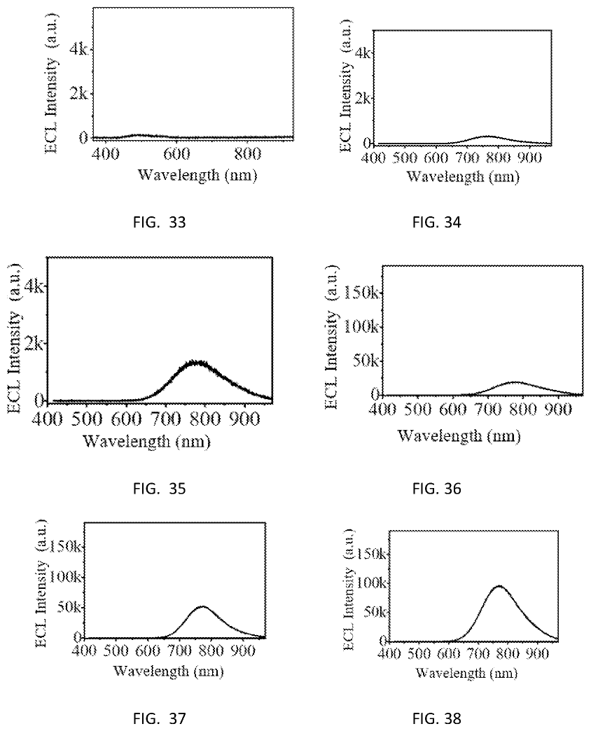

When the concentration of the CEA was 0, an ECL spectrogram of the ECL immunosensor with Au NCs as labels is shown in FIG. 33. The ECL immunosensor did not generate any ECL signal in 10 mM pH 7.4 Hepes buffer solution containing hydrazine hydrate.

### EXPERIMENT EXAMPLE 3

1. Establishment of an ECL sensor for nucleic acid testing with CIS@ZnS NCs as labels:
a cleaned Au electrode was soaked in 10 mM mercaptopropionic acid overnight, and MPA was bonded to the surface of the electrode through an Au—S bond;
10 μL of 10 mg/mL EDC and 10 mg/mL NHS were added dropwise to the surface of the modified electrode obtained in f, the mixture was activated for 30 min, the electrode was cleaned, and unreacted EDC and NHS were removed;
10 μL of 10 μM captured Cₚ₅₃ was added dropwise to the surface of the activated electrode obtained in g, incubated for 3 h and added to unreacted active sites on a BSA closed electrode, and the electrode was cleaned;
purified CIS@ZnS NCs were dissolved in a phosphate buffer solution containing EDC and NHS and activated, then Tₚ₅₃ was added, the mixture was incubated at a constant temperature of 37C for 3-5 h, BSA was added, and the mixture was sealed for 30 min to obtain Pₚ₅₃|CIS@ZnS NCs;
10 μL of Tₚ₅₃ of different concentrations was added dropwise to the surface of the electrode treated in h and incubated at room temperature for 90 min, the electrode was cleaned, and 10 μL of Pₚ₅₃|CIS@ZnS NCs was added dropwise to the surface of the electrode and incubated for 1 h; the Pₚ₅₃|CIS@ZnS NCs were grafted and fixed to the surface of the working electrode based on a form of immune complex formation to realize the preparation of an ECL sensor for nucleic acid testing with the maximum radiation wavelength at 775 nm.
   1) An ECL sensor for nucleic acid testing was prepared according to the steps described in Example 1, 10 μL of P₅₃ was added dropwise to the surface of the electrode treated in c, with T_{P53} concentrations of: 0.5 μM, 1 μM, 5 μM, 50 μM, 500 μM, 3,000 μM, 10,000 μM and 50,000 μM respectively to obtain an ECL sensor for nucleic acid testing with target DNA of different concentrations;
   2) The ECL sensor for nucleic acid testing was used as a working electrode, a platinum filament was used as a counter electrode, an Ag/AgCl electrode was used as a reference electrode, 10 mM pH 7.4 Hepes was used as a buffer solution, and 10 mM hydrazine hydrate was used as a coreactant, respectively. Cyclic voltammetry was used for driving, a potential window was 0-1.6 V, a scanning speed was 50 mV/s, an initial potential was 0 V, an initial scanning direction was positive, and an ECL spectrum obtained with target DNA of different concentrations is shown in FIGS. 34-41.
   3) All photons in the whole process of ECL were collected by means of exposure imaging, and a total spectrum was obtained by means of radiation of all the photons based on dispersive ECL; a working curve was drawn according to a relationship between light intensity at the maximum radiation wavelength on the spectral curve and the concentration of a standard solution of target DNA. The working curve of the ECL immunosensor for target DNA is shown in FIG. 42. An ECL signal was gradually enhanced as the target DNA concentration increased.

When the concentration of the target DNA T_{P53} was 0.5 μM, an ECL spectrogram of the ECL sensor for nucleic acid testing with CIS@ZnS NCs as labels is shown in FIG. 34. The ECL sensor could generate an ECL signal with the maximum emission wavelength at 775 nm in 10 mM pH 7.4 Hepes buffer solution containing hydrazine hydrate. Compared with the ECL spectrogram of the ECL sensor for synchronous implementation of immunoassay and nucleic acid testing in Example 1, the ECL signals at 775 nm were basically the same, and an immunoassay process and a nucleic acid testing process did not affect each other under this condition.

When the concentration of the target DNA DNA T_{P53} was 1 μM, an ECL spectrogram of the ECL sensor for nucleic acid testing with CIS@ZnS NCs as labels is shown in FIG. 35. The ECL sensor could generate an ECL signal with the maximum emission wavelength at 775 nm in 10 mM pH 7.4 Hepes buffer solution containing hydrazine hydrate. Compared with the ECL spectrogram of the ECL sensor for synchronous implementation of immunoassay and nucleic acid testing in Example 2, the ECL signals at 775 nm were basically the same, and an immunoassay process and a nucleic acid testing process did not affect each other under this condition.

When the concentration of the target DNA T_{P53} was 5 μM, an ECL spectrogram of the ECL sensor for nucleic acid testing with CIS@ZnS NCs as labels is shown in FIG. 36. The ECL sensor could generate an ECL signal with the maximum emission wavelength at 775 nm in 10 mM pH 7.4 Hepes buffer solution containing hydrazine hydrate. Compared with the ECL spectrogram of the ECL sensor for synchronous implementation of immunoassay and nucleic acid testing in Example 3, the ECL signals at 775 nm were basically the same, and an immunoassay process and a nucleic acid testing process did not affect each other under this condition.

When the concentration of the target DNA T_{P53} was 50 μM, an ECL spectrogram of the ECL sensor for nucleic acid testing with CIS@ZnS NCs as labels is shown in FIG. 37. The ECL sensor could generate an ECL signal with the maximum emission wavelength at 775 nm in 10 mM pH 7.4 Hepes buffer solution containing hydrazine hydrate. Compared with the ECL spectrogram of the ECL sensor for synchronous implementation of immunoassay and nucleic acid testing in Example 4, the ECL signals at 775 nm were basically the same, and an immunoassay process and a nucleic acid testing process did not affect each other under this condition.

When the concentration of the target DNA T_{P53} was 500 μM, an ECL spectrogram of the ECL sensor for nucleic acid testing with CIS@ZnS NCs as labels is shown in FIG. 38. The ECL sensor could generate an ECL signal with the maximum emission wavelength at 775 nm in 10 mM pH 7.4 Hepes buffer solution containing hydrazine hydrate. Compared with the ECL spectrogram of the ECL sensor for synchronous implementation of immunoassay and nucleic acid testing in Example 5, the ECL signals at 775 nm were basically the same, and an immunoassay process and a nucleic acid testing process did not affect each other under this condition.

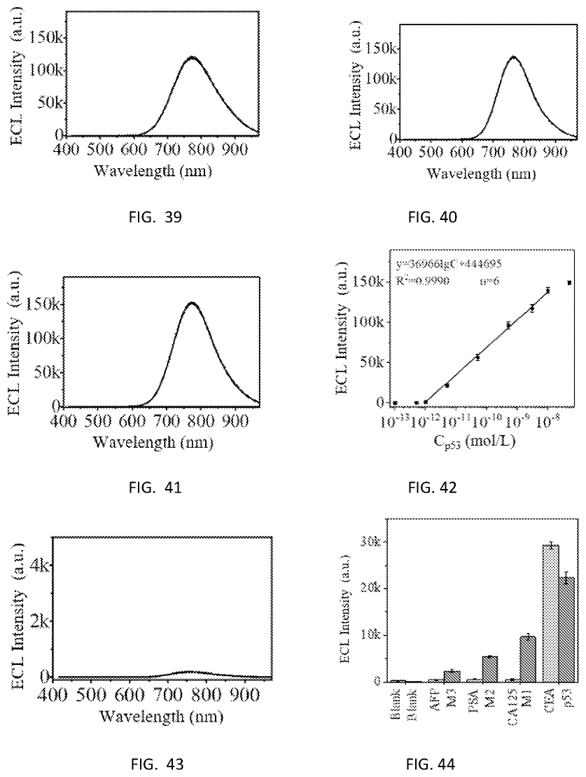

When the concentration of the target DNA T_{P53} was 3,000 μM, an ECL spectrogram of the ECL sensor for nucleic acid testing with CIS@ZnS NCs as labels is shown in FIG. 39. The ECL sensor could generate an ECL signal with the maximum emission wavelength at 775 nm in 10 mM pH 7.4 Hepes buffer solution containing hydrazine hydrate. Compared with the ECL spectrogram of the ECL sensor for synchronous implementation of immunoassay and nucleic acid testing in Example 6, the ECL signals at 775 nm were basically the same, and an immunoassay process and a nucleic acid testing process did not affect each other under this condition.

When the concentration of the target DNA T_{P53} was 10,000 μM, an ECL spectrogram of the ECL sensor for nucleic acid testing with CIS@ZnS NCs as labels is shown in FIG. 40. The ECL sensor could generate an ECL signal with the maximum emission wavelength at 775 nm in 10 mM pH 7.4 Hepes buffer solution containing hydrazine hydrate. Compared with the ECL spectrogram of the ECL sensor for synchronous implementation of immunoassay and nucleic acid testing in Example 7, the ECL signals at 775 nm were basically the same, and an immunoassay process and a nucleic acid testing process did not affect each other under this condition.

When the concentration of the target DNA T_{P53} was 50,000 μM, an ECL spectrogram of the ECL sensor for nucleic acid testing with CIS@ZnS NCs as labels is shown in FIG. 41. The ECL sensor could generate an ECL signal with the maximum emission wavelength at 775 nm in 10 mM pH 7.4 Hepes buffer solution containing hydrazine hydrate. Compared with the ECL spectrogram of the ECL sensor for synchronous implementation of immunoassay and nucleic acid testing in Example 8, the ECL signals at 775 nm were basically the same, and an immunoassay process and a nucleic acid testing process did not affect each other under this condition.

The working curve of the ECL immunosensor is shown in FIG. 42. An ECL signal was gradually enhanced as the target DNA concentration increased.

2. T_{P53} in Au|MPA-Cp53<Tₚ₅₃>Pp53|CIS@ZnS NCs was removed.

Au|MPA-Cₚ₅₃<Tₚ₅₃>Pₚ₅₃|CIS@ZnS NCs was used as a working electrode, a platinum filament was used as a counter electrode, an Ag/AgCl electrode was used as a reference electrode, 10 mM pH 7.4 Hepes was used as a buffer solution, and 10 mM hydrazine hydrate was used as a coreactant. Cyclic voltammetry was used for driving, a potential window was 0-1.6 V, a scanning speed was 50 mV/s, an initial potential was 0 V, an initial scanning direction was positive, and an ECL spectrum with different target DNA concentrations was obtained. However, the light intensities of the working curve of the ECL sensor for synchronous implementation of immunoassay and nucleic acid testing of the present invention under different concentrations were equivalent to that for separate testing. Judging from the ECL curves and spectra, each showed the same degree of decline after labeling, which proved that the immunoassay process and the nucleic acid testing process were basically not affected.

When the concentration of the target DNA was 0, an ECL spectrogram of the ECL sensor with CIS@ZnS NCs as labels is shown in FIG. 43, and the ECL sensor did not generate any ECL signal in 10 mM pH 7.4 Hepes buffer solution containing hydrazine hydrate.

Experiment Example 4 Specific testing:
The Ag (CEA) in step I in Example 1 was replaced with blank, alpha-fetoprotein antigen, prostate-specific antigen and carbohydrate antigen 125, respectively.

The T_{P53} was replaced with blank, single-base mismatched, double-base mismatched and three-base mismatched target DNAs.

A specific ECL response diagram of the established ECL sensor vs. antigen and target DNA is shown in FIG. 44. The ECL multicomponent analysis sensor prepared in this example had a good selectivity to the carcinoembryonic antigen, other antigen proteins did not interfere with the target antigen sensing of the invention, ECL signals were weakened with the increase of the number of mismatched bases, which showed that the ECL sensor had a good specificity for both carcinoembryonic antigen and target DNA.

### COMPARATIVE EXAMPLE 1

The differences from the immunoassay-nucleic acid testing synchronous ECL multicomponent analysis method of Example 1 were as follows:

The antigen in Au-MPA-Ab₁<Ag>Ab₂-AuNCs and the target DNA T_{P53} in Au|MPA-Cp53<Tₚ₅₃>Pₚ₅₃|CIS@ZnS NCs in step I were removed.

Au|MPA−Cₚ₅₃^{Ab1}<Tₚ₅₃^{Ag}>Pₚ₅₃^{Ab2}|CIS_{@ZnSNCs}^{Au NCs} was used as a working electrode, a platinum filament was used as a counter electrode, an Ag/AgCl electrode was used as a reference electrode, 10 mM pH 7.4 Hepes was used as a buffer solution, and 10 mM hydrazine hydrate was used as a coreactant. Cyclic voltammetry was used for driving, a potential window was 0-1.6 V, a scanning speed was 50 mV/s, an initial potential was 0 V, an initial scanning direction was positive, and an ECL spectrum with different target DNA concentrations was obtained.

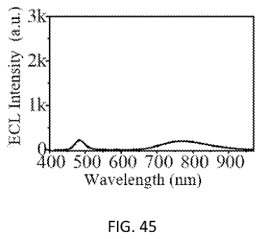

When the concentration of the CEA and that of the target DNA were 0, an ECL spectrogram of Au|MPA−Cₚ₅₃^{Ab1}<Tₚ₅₃^{Ag}>Pₚ₅₃^{Ab2}|CIS_{@ZnSNCs}^{Au NCs} is shown in FIG. 45. The ECL sensor did not generate any ECL signal in 10 mM pH 7.4 Hepes buffer solution containing hydrazine hydrate.

What is claimed is:

1. An ECL immunoassay-nucleic acid testing synchronous multicomponent analysis method based on spectral resolution principle, including the following steps:
i) establishing an ECL multicomponent analysis sensor for synchronous implementation of immunoassay and nucleic acid testing, wherein the maximum radiation wavelengths of the ECL multicomponent analysis sensor are at 485 nm and 775 nm respectively; the ECL multicomponent analysis sensor includes a secondary antibody labeled with Au NCs (Ab₂|Au NCs) and a probe DNA fragment labeled with CIS@ZnS NCs quantum dots (Pₚ₅₃|CIS@ZnS NCs), CEA-Ag and Tₚ₅₃, Au|MPA-Abᵢ and Au|MPA-Cₚ₅₃, and the ECL multicomponent analysis sensor Au|MPA−Cₚ₅₃^{Ab1}<Tₚ₅₃^{Ag}>Pₚ₅₃^{Ab2}|CIS_{@ZnSNCs}^{Au NCs} is established based on selectivity of immune and nucleic acid reactions;
ii) producing ECL by taking the ECL multicomponent analysis sensor as a working electrode, a platinum electrode as a counter electrode, and an Ag/AgCl electrode as a reference electrode when cyclic voltammetry is used for driving in a Hepes buffer solution containing 5-20 mM hydrazine hydrate;
iii) collecting all photons in the whole process of ECL by means of exposure imaging, and obtaining a total spectrum by means of radiation of all the photons based on dispersive ECL; drawing a working curve of CEA testing according to a relationship between the maximum radiation intensity at the maximum radiation wavelength of 485 nm on a spectral curve and the concentration of a standard antigen solution; drawing a working curve of Tₚ₅₃ testing according to a relationship between the maximum radiation intensity at the maximum radiation wavelength of 775 nm on the spectral curve and the concentration of Tₚ₅₃; and
iv) using a sample to be tested to establish an ECL sensor for synchronous implementation of immunoassay and nucleic acid testing according to step 1), performing an ECL spectral test according to the methods in step 2) and step 3), and synchronously testing the concentrations of the antigen and target DNA in the sample solution to be tested according to a light intensity signal and a working curve at the maximum radiation wavelength on the obtained ECL spectral curve.

2. The method according to claim 1, wherein in step ii) and step iv), when cyclic voltammetry scanning is performed, a scanning voltage ranges from 0 V to 1.6 V, a number of scanning turns is 1 to 3, and a scanning speed is 40 to 60 mV/s.

3. The method according to claim 1, wherein in step i), the method for establishing the ECL multicomponent analysis sensor for synchronous implementation of immunoassay and nucleic acid testing is as follows:
a. using a cleaned and activated Au electrode as a working electrode, and labeling both CEA-Ab₁ and Cₚ₅₃ on the surface of the electrode to obtain a double-labeled Au electrode;
b. labeling the water-soluble Au NCs with CEA-Ab₂ to obtain Ab₂|Au NCs; labeling the water-soluble CIS@ZnS NCs with a probe DNA to obtain Pₚ₅₃|CIS@ZnS NCs; and
c. adding CEA-Ag and Tₚ₅₃ dropwise to the surface of the double-labeled Au electrode, incubating the mixture at room temperature, adding the Ab₂|Au NCs and Pₚ₅₃|CIS@ZnS NCs obtained in step b dropwise to the surface of the electrode and incubating the mixture; grafting Ab₂|Au NCs and Pₚ₅₃|CIS@ZnS NCs to the surface of the working electrode in a form of immune complex formation to obtain an ECL multicomponent analysis sensor for synchronous implementation of immunoassay and nucleic acid testing.

4. The method according to claim 3, wherein in step a, the preparation steps of the double-labeled Au electrode are as follows:
(i) soaking a cleaned Au electrode in 5-20 mM mercaptopropionic acid overnight, and bonding MPA to the surface of the electrode through an Au—S bond;
(ii) adding 10 μL of 10 mg/mL 1-ethyl-(3-dimethylaminopropionic acid) carbodiimide hydrochloride (EDC) and 10 mg/mL hydroxysuccinimide (NHS) dropwise to the surface of the modified electrode obtained in step (1), activating the mixture for 30 min, cleaning the electrode, and removing unreacted EDC and NHS; and
(iii) mixing an aqueous solution of CEA-Ab₁ and an aqueous solution of Cₚ₅₃, adding the mixture to the surface of the activated electrode, incubating the mixture for 2-4 h, adding the mixture to unreacted active sites on a BSA closed electrode, and cleaning the electrode to obtain a double-labeled Au electrode; the concentration of the aqueous solution of CEA-Ab₁ is 8-15 g/mL with an added amount of 8-15 L, and the concentration of the aqueous solution of Cₚ₅₃ is 8-15 μM with an added amount of 8-15 μL.

5. The method according to claim 3, wherein in step b for establishing the ECL multicomponent analysis sensor for synchronous implementation of immunoassay and nucleic acid testing, the synthesis steps of the Ab₂|Au NCs are as follows:
i) activating carboxylic acid groups on the surface of water-soluble Au NCs; and
ii) enabling the secondary antibody to react with the carboxylic acid groups on the surface of the water-soluble Au NCs treated in step 1) to obtain a secondary antibody corresponding to the antigen labeled by the water-soluble Au NCs;
preferably, the specific preparation steps of the Ab₂|Au NCs are as follows:
dissolving purified Au NCs in 1 mL of 0.1 M pH 6.0 phosphate buffered solution (PBS) containing 10 mg/mL EDC and 10 mg/mL NHS, activating the mixture for 30 min, performing centrifugal purification, and dispersing the mixture in 1 mL of pH 7.4 0.1 M PBS to obtain activated Au NCs; adding 8-15 μL of 8-15 μg/mL aqueous solution of CEA-Ab₂, incubating the mixture at a constant temperature of 37° C. for 3-5 h, adding 20 μL of bovine serum albumin (BSA), sealing for 30 min, centrifuging the mixture, and collecting sediments to obtain Ab₂|Au NCs.

6. The method according to claim 5, wherein the above-mentioned water-soluble Au NCs are prepared from chloroauric acid as an Au source, mercaptopropionic acid as a stabilizer and zinc acetate as an aggregation inducer;
preferably, the synthesis steps of the water-soluble Au NCs are as follows:
(i) taking 35.5 μL of 100 mg/mL HAuCl₄-3H₂0, and adding 2.5 mL of deionized water;
(ii) adding 50 μL of mercaptopropionic acid to the mixture in step (i), and stirring the mixture for 15 min;
(iii) adding 430 μL of 1 M sodium hydroxide to the mixture in step (ii), and adjusting the pH to 8.5; and
(iv) adding 0.5 mL of 0.1 M zinc acetate to the mixture in step (3), stirring the mixture at room temperature for a reaction for 6 h, washing and purifying the reaction product with isopropanol after the reaction is completed to obtain water-soluble Au NCs.

7. The method according to claim 3, wherein in step b for establishing the ECL multicomponent analysis sensor for synchronous implementation of immunoassay and nucleic acid testing, the synthesis steps of the Pₚ₅₃|CIS@ZnS NCs are as follows:
i) activating carboxylic acid groups on the surface of water-soluble CIS@ZnS NCs; and
ii) making Pₚ₅₃ react with the carboxylic acid groups on the surface of the activated water-soluble CIS@ZnS NCs to obtain a probe DNA corresponding to a target DNA labeled with the water-soluble CIS@ZnS NCs;
preferably, the specific preparation steps of the Pₚ₅₃|CIS@ZnS NCs are as follows:
dissolving purified CIS@ZnS NCs in 1 mL of 0.1 M pH 6.0 PBS containing 10 mg/mL EDC and 10 mg/mL NHS, activating the mixture for 30 min, performing centrifugal purification, and dispersing the mixture in 1 mL of pH 7.4 0.1 M PBS to obtain activated CIS@ZnS NCs; adding 8-15 μL of 8-15 μM aqueous solution of Pₚ₅₃, incubating the mixture at a constant temperature of 37° C. for 3-5 h, connecting an amino group at one end of the probe DNA and a carboxyl group on the surface of CIS@ZnS NCs through an amidation reaction, adding 20 μL of BSA, sealing for 30 min, centrifuging the mixture, and collecting sediments to obtain Pₚ₅₃|CIS@ZnS NCs.

8. The method according to claim 7, wherein the above-mentioned water-soluble CIS@ZnS NCs are water-soluble CuInS₂@ZnS NCs prepared from CuCl₂ 2H₂0 as a Cu source, InCl₃ 4H₂0 as an In source, and sodium citrate and captopril as stabilizers;
preferably, the synthesis steps of the water-soluble CIS@ZnS NCs are as follows:
(i) dissolving 0.0022 g of captopril, 0.01 g of NaOH, 0.0471 g of sodium citrate, 0.0017 g of CuCl₂ 2H₂0, 0.0117 g of InCl₃ 4H₂0 and 0.0048 g of Na₂S in 20 mL of deionized water successively under a stirring condition, heating the mixture to 95° C., and holding for 45 min;
(ii) adding 0.177 g of Zn(CH₃COO)₂ and 0.061 g of thiourea to the mixture in step (i); and
(iii) adding isopropanol to the mixture in step (ii) for washing and purification to obtain water-soluble CIS@ZnS NCs.

9. The method according to claim 1, wherein ECL multicomponent analysis sensor for synchronous implementation of immunoassay and nucleic acid testing, is as follows:
a. soaking a cleaned Au electrode in 10 mM mercaptopropionic acid overnight, and bonding MPA to the surface of the electrode through an Au—S bond;
b. adding 10 μL of 10 mg/mL EDC and 10 mg/mL NHS dropwise to the surface of the modified electrode obtained in a, activating the mixture for 30 min, cleaning the electrode, and removing unreacted EDC and NHS;
c. mixing 10 μL of 10 μg/mL aqueous solution of CEA-Ab₁ and 10 μL of 10 μM aqueous solution of Cₚ₅₃, adding the mixture to the surface of the activated electrode obtained in step b, incubating the mixture for 3 h, adding the mixture to unreacted active sites on a BSA closed electrode, and cleaning the electrode to obtain a double-labeled Au electrode;
d. dissolving purified Au NCs in 1 mL of 0.1 M pH 6.0 PBS containing 10 mg/mL EDC and 10 mg/mL NHS, activating the mixture for 30 min, performing centrifugal purification, and dispersing the mixture in 1 mL of pH 7.4 0.1 M PBS to obtain activated Au NCs; adding 10 μL of 10 μg/mL aqueous solution of CEA-Ab₂, incubating the mixture at a constant temperature of 37° C. for 3-5 h, adding 20 μL of BSA, sealing for 30 min, centrifuging the mixture, and collecting sediments to obtain Ab₂|Au NCs;
e. dissolving purified CIS@ZnS NCs in 1 mL of 0.1 M pH 6.0 PBS containing 10 mg/mL EDC and 10 mg/mL NHS, activating the mixture for 30 min, performing centrifugal purification, and dispersing the mixture in 1 mL of pH 7.4 0.1 M PBS to obtain activated CIS@ZnS NCs; adding 10 μL of 10 μM aqueous solution of probe DNA, incubating the mixture at a constant temperature of 37C for 3-5 h, adding 20 μL of BSA, sealing for 30 min, centrifuging the mixture, and collecting sediments to obtain Pₚ₅₃|CIS@ZnS NCs; and
f. adding CEA-Ag and Tₚ₅₃ dropwise to the surface of the double-labeled Au electrode, incubating the mixture at room temperature for 90 min, cleaning the electrode, mixing and adding the Ab₂|Au NCs and Pₚ₅₃|CIS@ZnS NCs dropwise to the surface of the electrode and incubating the mixture for 1 h; grafting and fixing Ab₂|Au NCs and Pₚ₅₃|CIS@ZnS NCs in a form of immune complex formation to the surface of the working electrode to obtain an ECL sensor for synchronous implementation of immunoassay and nucleic acid testing;
when the above-mentioned added BSA is sealed, the volume fraction of BSA is 1%; flushing liquid used for cleaning the electrode is 10 mM pH=7.4 PBS;
the above-mentioned CEA-Ag and Tₚ₅₃ are added dropwise to the surface of the double-labeled Au electrode in the form of aqueous solutions, the concentration of CEA is 0.3 pg/mL 50 ng/mL, and the concentration of Tₚ₅₃ is 1 μM˜ 50 nM;
the Ab₂|Au NCs and Pₚ₅₃|CIS@ZnS NCs are added to the surface of the electrode dropwise in the form of aqueous solutions for incubation, and the concentration of Ab₂|Au NCs is 10-20 mg/mL; the concentration of Pₚ₅₃|CIS@ZnS NCs is 10-20 μM, and the amounts of Ab₂|Au NCs and Pₚ₅₃|CIS@ZnS NCs shall be sufficient; antigen-antibody interaction and complementary base pairing are formed.

10. The method according to claim 1, wherein specifically:
I. aqueous solutions of CEA-Ag with different standard concentrations and aqueous solutions of Tₚ₅₃ with different standard concentrations are prepared, the ECL sensor for synchronous implementation of immunoassay and nucleic acid testing is established according to the method for establishing an ECL sensor for synchronous implementation of immunoassay and nucleic acid testing using the aqueous solutions of CEA-Ag with different standard concentrations and the aqueous solutions of Tₚ₅₃ with different standard concentrations, and ECL is produced by taking the obtained sensor electrode as a working electrode, a platinum electrode as a counter electrode, and an Ag/AgCl electrode as a reference electrode when cyclic voltammetry is used for driving in a Hepes buffer solution containing 5-20 mM hydrazine hydrate;
II: collecting all photons in the whole process of ECL by means of exposure imaging, and obtaining a total spectrum by means of radiation of all the photons based on dispersive ECL; drawing a working curve of CEA testing according to a relationship between the maximum radiation intensity at the maximum radiation wavelength of 485 nm on a spectral curve and the concentration of standard antigens; drawing a working curve of Tₚ₅₃ testing according to a relationship between the maximum radiation intensity at the maximum radiation wavelength of 775 nm on the spectral curve and the concentration of Tₚ₅₃; and
III: an ECL sensor for synchronous implementation of immunoassay and nucleic acid testing is established according to the method for establishing an ECL sensor for synchronous implementation of immunoassay and nucleic acid testing using a target DNA to be tested and CEA-Ag to be tested; ECL is produced by taking the obtained sensor electrode as a working electrode, a platinum electrode as a counter electrode, and an Ag/AgCl electrode as a reference electrode when cyclic voltammetry is used for driving in a Hepes buffer solution containing 5-20 mM hydrazine hydrate; the concentrations of the antigen and target DNA in the sample solution to be tested are synchronously tested according to a light intensity signal and a working curve at the maximum radiation wavelength on the obtained ECL spectral curve.
